(19) United States
(12) Patent Application Publication
(10) Pub. No.: US 2006/0015178 A1
(43) Pub. Date: Jan. 19, 2006

(54) Implants and methods for reshaping heart valves

(76) Inventors: Shahram Moaddeb, Irvine, CA (US); Emanuel Shaoulian, Newport Beach, CA (US); Samuel M. Shaolian, Newport Beach, CA (US); Michael R. Henson, Coto de Caza, CA (US); Richard S. Rhee, Anaheim, CA (US); Steven C. Anderson, Rancho Santa Margarita, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/181,686

(22) Filed: Jul. 14, 2005

Related U.S. Application Data
(60) Provisional application No. 60/588,253, filed on Jul. 15, 2004.

(51) Int. Cl. A61F 2/24

(52) U.S. Cl. 623/2.36; 606/151; 600/12

(57) ABSTRACT

Tissue shaping methods and devices are provided for reinforcing and/or remodeling heart valves. In certain embodiments, magnetic tissue shaping devices are implanted in tissue adjacent heart valve leaflets. The devices are mutually attractive or repulsive so as to remodel the heart tissue and improve heart valve function. In certain other embodiments, one or more tissue shaping devices including shape memory material are implanted in a patient's body within or on tissue adjacent a heart valve leaflet. The shape memory material can be activated within the patient in a less invasive or non-invasive manner, such as by applying energy percutaneously or external to the patient's body. The shape memory tissue shaping devices are implanted in a first configuration and then activated to remember a second configuration that displaces tissue so as to remodel the heart valve geometry and improve heart valve function. In certain other embodiments, a brace is crimped to the base of a heart valve leaflet to support the leaflet and improve valve closure.

### RELATED APPLICATIONS

This application claims the benefit under 35 U.S.C. § 119(e) of U.S. Provisional Application No. 60/588,253, filed Jul. 15, 2004, the entirety of which is hereby incorporated by reference herein.

### BACKGROUND OF THE INVENTION

1. Field of the Invention

The present invention generally relates to implants and methods for reshaping tissue and, more specifically, for reshaping and resizing dysfunctional heart valves.

2. Description of the Related Art

The circulatory system of mammals includes the heart and the interconnecting vessels throughout the body that include both veins and arteries. The human heart includes four chambers, which are the left and right atrium and the left and right ventricles. The mitral valve, which allows blood flow in one direction, is positioned between the left ventricle and left atrium. The tricuspid valve is positioned between the right ventricle and the right atrium. The aortic valve is positioned between the left ventricle and the aorta, and the pulmonary valve is positioned between the right ventricle and pulmonary artery. The heart valves function in concert to move blood throughout the circulatory system. The right ventricle pumps oxygen-poor blood from the body to the lungs and then into the left atrium. From the left atrium, the blood is pumped into the left ventricle and then out the aortic valve into the aorta. The blood is then recirculated throughout the tissues and organs of the body and returns once again to the right atrium.

If the valves of the heart do not function properly, due either to disease or congenital defects, the circulation of the blood may be compromised. Diseased heart valves may be stenotic, wherein the valve does not open sufficiently to allow adequate forward flow of blood through the valve, and/or incompetent, wherein the valve does not close completely. Incompetent heart valves cause regurgitation or excessive backward flow of blood through the valve when the valve is closed. For example, certain diseases of the heart valves can result in dilation of the heart and one or more heart valves. When a heart valve annulus dilates, the valve leaflet geometry deforms and causes ineffective closure of the valve leaflets. The ineffective closure of the valve can cause regurgitation of the blood, accumulation of blood in the heart, and other problems.

Mitral valve regurgitation is a common type of heart valve insufficiency and can be one of the main contributors to heart deterioration and failure. Mitral valve regurgitation is a serious, often rapidly deteriorating, condition that reduces circulatory efficiency. Oftentimes, mitral regurgitation is caused by geometric changes of the left ventricle, papillary muscles and mitral annulus. Weakened mitral valves that allow regurgitation can protrude into the left atrium, a condition known as mitral valve prolapse.

Diseased or damaged heart valves can be treated by valve replacement surgery, in which damaged leaflets are excised and the annulus is sculpted to receive a replacement valve. Another repair technique that has been shown to be effective in treating incompetence is annuloplasty, in which the effective size of the valve annulus is contracted by attaching a prosthetic annuloplasty repair segment or ring to an interior wall of the heart around the valve annulus. The annuloplasty ring reinforces the functional changes that occur during the cardiac cycle to improve coaptation and valve integrity. Thus, annuloplasty rings help reduce reverse flow or regurgitation while permitting good hemodynamics during forward flow.

Each of these procedures, however, is highly invasive because access to the heart is obtained through an open chest procedure wherein a heart-lung machine bypasses the heart throughout the procedure. Most patients with mitral valve regurgitation, however, are often relatively frail, thereby increasing the risk associated with such an operation.

### SUMMARY OF THE INVENTION

In view of the foregoing, conventional systems and methods for treating valvular insufficiency do not provide for a less invasive approach that reduces strain on the patient. A need, therefore, remains for devices and methods for supporting heart valves or other body structures that can be safely and reliably deployed and adapted to the dynamic environment of a human or animal cardiac system. Thus, it would be advantageous to develop devices and methods that allow for non-invasive adjustment of an implant usable to treat valvular insufficiency such as mitral valve insufficiency. Furthermore, a need exists for an implant that may be non-invasively adjusted after implantation into a patient.

In one embodiment, an implant for reinforcing a patient's heart valve includes a body member having a proximal end, a distal end and a length extending therebetween. The body member is configured to be implanted within a patient's heart at or near a base of a heart valve leaflet. The body member comprises a shape memory material and is transformable from a first configuration to a second configuration. When the body member is in the second configuration, the body member is configured to reshape a tissue of the heart so as to exert a force on the leaflet base. The implant is elongate with its longest length less than or equal to about fifteen millimeters. In certain other embodiments, the longest length of the implant is less than or equal to about ten millimeters. In yet other embodiments, the longest length of the implant is less than or equal to about six millimeters.

In certain embodiments, the body member is substantially straight when in the first configuration and is substantially arcuate when in the second configuration. The implant is implanted within the patient's heart when the body member is in the first configuration. In certain embodiments, the implant is configured to be implanted wholly within the tissue of the heart. In certain other embodiments, the implant is configured to be positioned adjacent a surface of the tissue of the heart and may include one or more anchor members configured to securely attach the body member to the surface of the tissue of the heart.

The heart tissue may include, for example, myocardium, the interventricular septum of the heart, a fibrous trigone, a wall of an atrium, or other heart tissue. In certain embodiments, the implant is configured to be deliverable by a retrograde delivery system utilizing a retrograde approach into the left ventricle of the patient's heart when the body member is in the first configuration. In other embodiments, the implant is configured to be deliverable by a transseptal delivery system utilizing a transseptal approach into the left atrium of the patient's heart when the body member is in the first configuration.

In certain embodiments, the shape memory material is configured to be superelastic in at least one of the first configuration and the second configuration. The shape memory material may include, for example, a shape memory alloy, a shape memory polymer, or other material. In certain other embodiments, the shape memory material is ferromagnetic material and includes at least one of Fe—C, Fe—Pd, Fe—Mn—Si, Co—Mn, Fe—Co—Ni—Ti, Ni—Mn—Ga, Ni₂MnGa, and Co—Ni—Al. In certain such embodiments, the body member is configured to transform from the first configuration to the second configuration without substantially changing the temperature of the ferromagnetic shape memory material.

In certain embodiments, the body member is configured to transform from the first configuration to the second configuration when the shape memory material is activated by an energy source. The energy source may include, for example, an ultrasound energy source. In certain embodiments, the implant further comprising an energy absorption enhancement material configured to absorb energy and heat in response to the energy source, the energy absorption enhancement material in thermal communication with the shape memory material. The energy absorption enhancement material may include, for example, a nanoparticle comprising at least one of a nanoshell and a nanosphere. In certain embodiments, the energy absorption enhancement material is radiopaque. In certain embodiments, the implant also includes an electrically conductive material configured to conduct a current in response to the energy source and to transfer thermal energy to the shape memory material.

In one embodiment, an implant for reinforcing a patient's heart valve includes a body member having a proximal end, a distal end and a length extending therebetween. The body member comprises a shape memory material and is transformable from a first configuration to a second configuration. When the body member is in the second configuration, the body member is configured to reshape a tissue of the heart so as to exert a force on the leaflet base. The implant is elongate and is configured to be wholly implanted within the heart tissue. In certain such embodiments, the implant is substantially straight when the body member is in the first configuration and has a substantially arcuate shape when the body member is in the second configuration. The implant is implanted within the patient's heart when the body member is in the first configuration. In certain such embodiments, the longest length of the implant is less than or equal to about fifteen millimeters. In other embodiments, the longest length of the implant is less than or equal to about ten millimeters. In certain other embodiments the longest length of the implant is less than or equal to about six millimeters. In certain embodiments, the shape memory material is configured to be superelastic in at least one of the first configuration and the second configuration.

In one embodiment, a method of treating heart valve disease includes providing an implant comprising a body member having a proximal end, a distal end and a length extending therebetween, wherein the body member comprises a shape memory material. The method also includes wholly implanting the implant within a tissue of a patient's heart at or near a base of a valve leaflet, and applying energy to the shape memory material so as to transform the implant from a first configuration having a first shape to a second configuration having a second shape. The implant in the second configuration reshapes tissue adjacent the implant and produces a change in a dimension of the annulus of the valve. In certain such embodiments, the change in dimension urges the base of the leaflet toward the center of the heart valve. In certain such embodiments, applying the energy comprises applying the energy with an energy source located outside the patient's heart and unattached to the implant. In certain embodiments, positioning the implant comprises delivering the implant using a retrograde approach through the patient's aorta into the left ventricle of the patient's heart. In certain other embodiments, positioning the implant comprises delivering the implant using a transseptal approach into the left atrium of the patient's heart.

In one embodiment, a device for reshaping or reforming body tissue includes resilient means for changing a dimension of a heart valve annulus. The resilient means is configured to be implanted at or near the base of a leaflet of a patient's heart valve. The resilient means is also configured to transform from a first shape to a second shape in response to a force applied thereto during implantation. The resilient means transforms back to the first shape when the force is removed therefrom after the implantation. In certain such embodiments, the resilient means is configured to be wholly implanted within a tissue of the heart.

In one embodiment, a method for changing a dimension of a heart valve annulus includes implanting a first device and a second device in a patient's heart. The first device is magnetic and the second device is responsive to a magnetic field emanating from the first device so as to produce a change in a dimension of a heart valve annulus. In certain such embodiments, the change in the dimension comprises a decrease. In certain embodiments, the second device is magnetic and the magnetic field is a first magnetic such that the first device is responsive to a second magnetic field emanating from the second device so as to further produce the change in the dimension of the heart valve annulus. In certain embodiments, the first device is implanted adjacent a first leaflet of the heart valve and the second device is implanted adjacent a second leaflet of the heart valve such that the second device's response to the magnetic field urges the base of the second leaflet toward the base of the first leaflet. In other embodiments, the first device is implanted on the atrial side of the heart valve annulus adjacent a first leaflet thereof and the second device is implanted on the ventricular side of the heart valve annulus adjacent the first leaflet, and the second device's response to the magnetic field urges the base of the first leaflet toward a base of a second leaflet of the heart valve.

In one embodiment, a tissue shaping system includes a first device configured to emanate a magnetic field. The first device is configured to be implanted at or near a heart valve annulus. The tissue shaping system also includes a second device configured to interact with the first device by responding to the magnetic field. The second device is configured to be implanted at or near the heart valve annulus. The first device is configured to interact with the second device so as to change a dimension of the heart valve annulus. In certain such embodiments, the second device is also magnetic and the interaction between the first device and the second device is an attraction. In certain embodiments, the first device and the second device are configured to exert at least one force sufficient to decrease the dimension of the heart valve annulus when the first device and the second device are implanted adjacent thereto. In certain embodiments, the first device comprises a rare earth element. In certain embodiments, the first device comprises at least one of the following: NdFeB (Neodymium Iron Boron), SmCo (Samarium Cobalt) and AlNiCo (Aluminum Nickel Cobalt). In certain embodiments, at least one fixation member is configured to anchor at least one of the first device and the second device to the heart valve annulus.

In one embodiment, a system for reshaping or reforming a heart valve annulus includes means for emanating a magnetic field, and means for interacting with the means for emanating by responding to the magnetic field. The means for emanating and the means for interacting are implanted at or near the heart valve annulus. At least one dimension of the heart valve annulus is changed while the means for interacting responds to the magnetic field.

In one embodiment, a device for treating a defective heart valve comprising a ring-like member attachable to a heart valve leaflet. In certain such embodiments, the ring-like member comprises one or more of the following materials: stainless steel, NiTi, platinum iridium, gold, carbon, and polyurethane. The ring-like member is configured to provide rigid mechanical strength and support to the leaflet and may be crimped into place around the leaflet. In certain embodiments, the ring-like member further comprises one or more resilient axial extensions extending axially from the crimpable ring. In certain such embodiments, the one or more resilient axial extensions are configured to urge the inward end of the leaflet toward the center of the heart valve for improved coaptation with another leaflet. In certain embodiments, the one or more resilient axial extensions comprise carbon fiber.

In one embodiment, a method of supporting a heart valve leaflet includes providing an implant comprising a ring-like member and sliding the implant around the heart valve leaflet. In certain such embodiments, the method further includes crimping the ring-like member to secure it to the heart valve leaflet. In certain embodiments, the implant further comprises one or more resilient axial extensions, and sliding the implant around the heart valve leaflet further comprises sliding the implant such that the one or more resilient axial extensions extend away from the ring-like member toward the inward end of the leaflet. In certain embodiments, sliding the implant around the heart valve leaflet comprises delivering the implant using a retrograde approach into the left ventricle of the patient's heart. In certain other embodiments, sliding the implant around the heart valve leaflet comprises delivering the implant using a transseptal approach into the left atrium of the patient's heart.

In one embodiment, a device for improving leaflet coaptation in a heart valve includes means for supporting the leaflet. In certain such embodiments, the means for supporting is crimpable. In certain embodiments, the means for supporting the leaflet comprises means for urging the inward end of the leaflet toward another leaflet.

For purposes of summarizing the invention, certain aspects, advantages and novel features of the invention have been described herein. It is to be understood that not necessarily all such advantages may be achieved in accordance with any particular embodiment of the invention. Thus, the invention may be embodied or carried out in a manner that achieves or optimizes one advantage or group of advantages as taught herein without necessarily achieving other advantages as may be taught or suggested herein.

### BRIEF DESCRIPTION OF THE DRAWINGS

FIG. 1 is a cross-sectional view of a human heart, with some features of the heart not shown for clarity.

FIG. 2 is a cross-sectional view of the left ventricle of the heart shown in FIG. 1 illustrating regurgitation of blood back into the left atrium during systole due to defective closure of the leaflets of the mitral valve.

FIGS. 3A and 3B are cross-sectional views of the left ventricle of the heart shown in FIG. 1 illustrating a plurality of magnetic tissue shaping devices implanted in myocardial tissue adjacent the mitral valve leaflets according to certain embodiments of the invention.

FIGS. 4A and 4B are cross-sectional views of the left ventricle of the heart shown in FIG. 1 illustrating two mutually attractive magnetic tissue shaping devices implanted on opposite sides of the plane of the mitral valve according to certain embodiments of the invention.

FIGS. 5A-5C are cross-sectional views of the left ventricle of the heart shown in FIG. 1 illustrating two tissue shaping devices comprising a shape memory material deployed in myocardial tissue adjacent the leaflets of the mitral valve according to certain embodiments of the invention.

FIGS. 6A-6D schematically illustrate exemplary embodiments of the tissue shaping device capable of transforming from a first configuration to a second configuration according to certain embodiments of the invention.

FIG. 7 is a cross-sectional view of the left ventricle of a heart illustrating a plurality of tissue shaping devices comprising a shape memory material implanted within the mitral valve annulus according to certain embodiments of the invention.

FIGS. 8A and 8B illustrate top schematic views of four tissue shaping devices implanted in a mitral valve annulus according to an exemplary embodiment of the invention.

FIG. 9 is a schematic diagram illustrating a tissue shaping device including a shape memory body member defining one or more fixation anchors on a surface thereof according to certain embodiments of the invention.

FIG. 10 is a cross sectional view of the left ventricle of a heart illustrating a plurality of tissue shaping devices implanted on and/or adjacent to the surface of the mitral valve annulus according to certain embodiments of the invention.

FIG. 11 schematically illustrates an exemplary embodiment of a tissue shaping device that is dynamically adjustable to effect changes in at least one dimension of a mitral valve annulus.

FIG. 12 schematically illustrates another exemplary embodiment of a tissue shaping device that is dynamically adjustable to effect changes in the shape of the mitral valve annulus.

FIG. 13A is a perspective view of a portion of a tissue shaping device comprising a shape memory wire according to certain embodiments of the invention.

FIG. 13B is a perspective view of a portion of a tissue shaping device comprising a first wire and a second wire according to certain embodiments of the invention.

FIGS. 14A and 14B schematically illustrate a tissue shaping device including a shape memory wire substantially coated with an energy absorption layer according to certain embodiments of the invention.

FIGS. 15A-15C schematically illustrate a tissue shaping device including an electrically conductive coil according to certain embodiments of the invention.

FIG. 16 is a cross-sectional view of the human heart shown in FIG. 1 and a distal portion of a transseptal delivery system using a transseptal approach to deliver the tissue shaping devices according to certain embodiments of the invention.

FIG. 17 is a cross-sectional view of the human heart shown in FIG. 1 and a distal portion of a retrograde delivery system using a retrograde approach to deliver tissue shaping devices to myocardial tissue on the ventricular side of the mitral valve according to certain embodiments of the invention.

FIG. 18 is a cross-sectional view of the left ventricle of the heart shown in FIG. 1 wherein the leaflets of the mitral valve are deformed such that proper sealing and valve function is impeded.

FIG. 19 illustrates leaflet braces deployed over and crimped to the leaflets of the mitral valve shown in FIG. 18.

FIGS. 20A and 20B schematically illustrate the leaflet brace shown in FIG. 19 according to certain embodiments of the invention.

FIG. 21 schematically illustrates a transverse cross-section of a leaflet brace according to other embodiments of the invention.

FIG. 22 is a cross-sectional view of the left ventricle shown in FIG. 18 illustrating leaflet braces deployed over and crimped to the leaflets of the mitral valve according to certain embodiments of the invention.

FIGS. 23A and 23B schematically illustrate the leaflet brace shown in FIG. 22 according to certain embodiments of the invention.

FIG. 24 schematically illustrates a transverse cross-section of a leaflet brace 2400 according to other embodiments of the invention.

FIG. 25 illustrates a schematic view of an external source usable outside a patient's body to adjust a tissue shaping device positioned within the patient's heart according to certain embodiments of the invention.

FIG. 26 is a partial cross-sectional view of a catheter configured to deliver a resilient tissue shaping device according to certain embodiments of the invention.

FIG. 27A illustrates a top schematic view of a plurality of resilient tissue shaping devices implanted in a mitral valve annulus according to an exemplary embodiment of the invention.

FIG. 27B schematically illustrates a resilient tissue shaping devices being implanted in the mitral valve annulus shown in FIG. 27A through the distal end of the catheter shown in FIG. 26.

### DETAILED DESCRIPTION OF THE PREFERRED EMBODIMENT

Overview

The present invention involves devices and methods to reshape tissue, such as by reinforcing dysfunctional heart valves and other body tissue through a dynamically adjustable implant. Although embodiments of the invention disclosed herein are described with reference to the reshaping and/or resizing of a mitral valve of a human heart, embodiments of the invention may also be used with a wide variety of other valves, vessels, and/or tissue that require reshaping or reforming. For example, certain embodiments may be used to change at least one dimension of the tricuspid valve, the pulmonary valve, or the aortic valve. In other embodiments, tissue shaping devices may be used to reshape or reform left or right ventricles, gastric system tissue and/or organs (e.g., stomach), or the like.

In certain embodiments, methods of providing support to a defective heart valve structure include deploying a first field producing member in heart tissue adjacent a base of a leaflet of the heart valve and deploying a second field producing member in heart tissue such that the second field producing member is mutually attracted to the first field producing member so as to reduce a dimension of the heart valve. In certain such embodiments, multiple magnetic structures are implanted adjacent or within the heart valve annulus.

In other embodiments, a method of providing support to a defective heart valve structure includes deploying a shape memory member in heart tissue adjacent a base of a leaflet of the heart valve and activating the shape memory member to remember a configuration or shape that expands tissue immediately adjacent the shape memory member so as to force the leaflet base in an inward radial direction. In certain such embodiments, one or more shape memory members are implanted in the heart valve annulus. In other embodiments, a method of supporting the base of a heart valve leaflet includes deploying a crimpable ring about the base of the leaflet and crimping the ring in place on the leaflet to support the base of the leaflet and improve valve function.

In certain embodiments, a dynamically adjustable tissue shaping device is used to reshape and resize the mitral valve annulus via implanting the device adjacent to or within the mitral valve annulus. In particular, the tissue shaping device is used to dynamically change at least one dimension of the mitral valve annulus to improve leaflet coaptation and to reduce regurgitation. After implantation, the shape of the tissue shaping device can be further adjusted to compensate for changes in the size of the heart. For example, the tissue shaping device may be implanted in a child whose heart grows as the child gets older. Thus, the shape of the tissue shaping device may need to be modified to allow for expansion of the heart. As another example, the size of an enlarged heart may start to return to its normal size after implantation of one or more tissue shaping devices. Thus, the shape of the tissue shaping device may need to be modified to continue to reinforce the mitral valve annulus after the size of the heart size has been reduced.

In certain embodiments, the tissue shaping device comprises a shape memory material that is responsive to changes in temperature and/or exposure to a magnetic field. Shape memory is the ability of a material to regain or return to a particular shape after deformation. Shape memory materials include, for example, polymers, metals, metal alloys and ferromagnetic alloys. In certain embodiments, the tissue shaping device is adjusted in vivo by applying an energy source to activate the shape memory material and cause it to change to a memorized or prior shape. The energy source may include, for example, radio frequency (RF) energy, x-ray energy, microwave energy, acoustic or ultrasonic energy such as focused ultrasound or high intensity focused ultrasound (HIFU) energy, light energy, electric field energy, magnetic field energy, combinations of the same, or the like. For example, one embodiment of electromagnetic radiation may include infrared energy having a wavelength in a range between approximately 750 nanometers and approximately 1600 nanometers. This type of infrared radiation may be produced by a solid state diode laser.

In certain embodiments, the tissue shaping device further includes an energy absorbing material to increase heating efficiency and substantially localize heating in a select area of the shape memory material. Thus, damage to the surrounding tissue is reduced or minimized. Energy absorbing materials for light or laser activation energy may include nanoshells, nanospheres and the like, particularly where infrared laser energy is used to energize the material. Such nanoparticles may be made from a dielectric, such as silica, coated with an ultra thin layer of a conductor, such as gold, and may be selectively tuned to absorb a particular frequency of electromagnetic radiation. In certain such embodiments, the nanoparticles range in size between about 5 nanometers and about 20 nanometers and can be suspended in a suitable material or solution, such as a saline solution. Coatings comprising nanotubes or nanoparticles may also be used to absorb energy from, for example, HIFU, MRI, inductive heating or the like.

In certain embodiments, thin film deposition or other coating techniques such as sputtering, reactive sputtering, metal ion implantation, physical vapor deposition, and chemical deposition can be used to cover portions or all of the tissue shaping device. Such coatings can be either solid or microporous. When HIFU energy is used, for example, a microporous structure traps and directs the HIFU energy toward the shape memory material. The coating improves thermal conduction and heat removal. In certain embodiments, the coating also enhances radio-opacity of the tissue shaping device. Coating materials can be selected from various groups of biocompatible organic or non-organic, metallic or non-metallic materials such as Titanium Nitride (TiN), Iridium Oxide (Irox), Carbon, Platinum black, Titanium Carbide (TiC) and other materials used for pacemaker electrodes for implantable pacemaker leads. Other materials discussed herein or known in the art can also be used to absorb energy.

In addition, or in other embodiments, fine conductive wires such as platinum coated copper, titanium, tantalum, stainless steel, gold, or the like, are wrapped around the shape memory material to allow focused and rapid heating of the shape memory material while reducing undesired heating of surrounding tissues.

In certain embodiments, the energy source is applied surgically either during or after implantation. For example, the shape memory material may be heated during implantation of the tissue shaping device by touching the tissue shaping device, or surrounding area, with a warm object or fluid. As another example, the energy source may be surgically applied after the tissue shaping device has been implanted, such as by percutaneously inserting a catheter into the patient's body and applying the energy through the catheter. For example, RF energy, light energy or thermal energy (e.g., from a heating element using resistance heating) can be transferred to the shape memory material through a catheter positioned on or near the shape memory material.

Alternatively, thermal energy can be provided to the tissue shaping device by injecting a heated fluid through a catheter or by circulating the heated fluid in a balloon through the catheter placed in close proximity to the tissue shaping device. As another example, the shape memory material can be coated with a photodynamic absorbing material which is activated to heat the shape memory material when illuminated by light from a laser diode or directed to the coating through fiber optic elements in a catheter. In certain such embodiments, the photodynamic absorbing material includes one or more drugs that are released when illuminated by the laser light.

In certain embodiments, a removable subcutaneous electrode or coil couples energy from a dedicated activation unit. In certain such embodiments, the removable subcutaneous electrode provides telemetry and power transmission between the system and the tissue shaping device. The subcutaneous removable electrode allows more efficient coupling of energy to the implant with minimum or reduced power loss. In certain embodiments, the subcutaneous energy is delivered via inductive coupling.

In other embodiments, the energy source is applied in a non-invasive, or less invasive, manner from outside the patient's body. In certain such embodiments, the external energy source may be focused to provide directional heating to the shape memory material to reduce or minimize damage to the surrounding tissue. For example, in certain embodiments, a portable device comprising an electrically conductive coil generates an electromagnetic field that non-invasively penetrates the patient's body and induces a current in the tissue shaping device. The current heats the tissue shaping device and causes the shape memory material to transform to a memorized shape. In certain such embodiments, the tissue shaping device also comprises an electrically conductive coil wrapped around or embedded in the memory shape material. The externally generated electromagnetic field induces a current in the tissue shaping device's coil, thereby causing it to heat and transfer thermal energy to the shape memory material.

In certain other embodiments, an external transducer focuses ultrasound energy onto the implanted tissue shaping device to heat the shape memory material. The term “focused ultrasound” as used herein is a broad term and is used in its ordinary sense and includes, without limitation, acoustic energy within a wide range of intensities and/or frequencies. For example, focused ultrasound energy includes high intensity focused ultrasound (HIFU) energy and/or acoustic energy having an intensity and/or frequency that is considerably less than what is currently used for ablation in medical procedures.

For instance, in certain embodiments, focused ultrasound energy includes acoustic energy within a frequency range of approximately 0.5 MHz to approximately 30 MHz and a power density within the range of approximately 1 W/cm² and approximately 500 W/cm². In further embodiments, focused ultrasound energy includes an intensity of acoustic energy that results in non-destructive heating such that little or no tissue damage occurs from the heating and/or such that effects from cavitation are reduced or substantially eliminated.

For exemplary purposes, the term HIFU is used herein with respect to certain embodiments of the invention. However, it is to be understood that other intensities of focused ultrasound energy, and in particular, relatively low intensities of focused ultrasound energy, may advantageously be used in place of, or in combination with, HIFU energy.

In certain embodiments, a HIFU probe is used with an adaptive lens to compensate for heart and respiration movement. The adaptive lens has multiple focal point adjustments. In certain embodiments, a HIFU probe with adaptive capabilities comprises a phased array or linear configuration. In certain embodiments, an external HIFU probe comprises a lens configured to be placed between a patient's ribs to improve acoustic window penetration and reduce or minimize issues and challenges regarding passing through bones. In certain embodiments, HIFU energy is synchronized with an ultrasound imaging device to allow visualization of the tissue shaping device during HIFU activation. In addition, or in other embodiments, ultrasound imaging is used to non-invasively monitor the temperature of tissue surrounding the tissue shaping device by using principles of speed of sound shift and changes to tissue thermal expansion.

In certain embodiments, the tissue shaping device comprises an ultrasound absorbing material or hydro-gel material that allows focused and rapid heating when exposed to the ultrasound energy and transfers thermal energy to the shape memory material.

In certain embodiments, non-invasive energy is applied to the implanted tissue shaping device using a Magnetic Resonance Imaging (MRI) device. In certain such embodiments, the shape memory material is activated by a constant magnetic field generated by the MRI device. In addition, or in other embodiments, the MRI device generates RF pulses that induce current in the tissue shaping device and heat the shape memory material. The tissue shaping device can include one or more coils and/or MRI energy absorbing material to increase the efficiency and directionality of the heating. Suitable energy absorbing materials for magnetic activation energy include particulates of ferromagnetic material. Suitable energy absorbing materials for RF energy include ferrite materials as well as other materials capable of absorbing RF energy at resonant frequencies thereof.

In certain embodiments, the MRI device is further used to determine the size and/or shape of the implanted tissue shaping device before, during and/or after the shape memory material is activated. In certain such embodiments, the MRI device generates RF pulses at a first frequency to heat the shape memory material and at a second frequency to image the implanted tissue shaping device. Thus, the size and/or shape of the tissue shaping device can be measured without heating the device. In certain such embodiments, an MRI energy absorbing material heats sufficiently to activate the shape memory material when exposed to the first frequency and does not substantially heat when exposed to the second frequency. Other imaging techniques known in the art can also be used to determine the size of the implanted device including, for example, ultrasound imaging, computed tomography (CT) scanning, X-ray imaging, position emission tomography (PET) or the like. In certain embodiments, such imaging techniques also provide sufficient energy to activate the shape memory material.

In certain embodiments, activation of the shape memory material is synchronized with the heart beat during an imaging procedure. For example, an imaging technique can be used to focus HIFU energy onto a tissue shaping device in a patient's body during a portion of the cardiac cycle. As the heart beats, the tissue shaping device may move in and out of this area of focused energy. To reduce damage to the surrounding tissue, the patient's body is exposed to the HIFU energy only during select portions of the cardiac cycle. In certain embodiments, the energy is gated with a signal that represents the cardiac cycle, such as an electrocardiogram signal. In certain such embodiments, the synchronization and gating is configured to allow delivery of energy to the shape memory materials at specific times during the cardiac cycle to avoid or reduce the likelihood of causing arrhythmia or fibrillation during vulnerable periods. For example, the energy can be gated so as to only expose the patient's heart to the energy during the T wave of the electrocardiogram signal.

As discussed above, shape memory materials include, for example, polymers, metals, and metal alloys including ferromagnetic alloys. Exemplary shape memory polymers that are usable for certain embodiments of the present invention are disclosed by Langer, et al. in U.S. Pat. No. 6,720,402, issued Apr. 13, 2004, U.S. Pat. No. 6,388,043, issued May 14, 2002, and U.S. Pat. No. 6,160,084, issued Dec. 12, 2000, each of which is hereby incorporated herein by reference in its entirety.

Shape memory polymers respond to changes in temperature by changing to one or more permanent or memorized shapes. In certain embodiments, the shape memory polymer is heated to a temperature between approximately 38° C. and approximately 60° C. In certain other embodiments, the shape memory polymer is heated to a temperature in a range between approximately 40° C. and approximately 55° C. In certain embodiments, the shape memory polymer has a two-way shape memory effect, wherein the shape memory polymer is heated to change it to a first memorized shape and cooled to change it to a second memorized shape. The shape memory polymer can be cooled, for example, by inserting or circulating a cooled fluid through a catheter.

Shape memory polymers implanted in a patient's body can be heated non-invasively using, for example, external light energy sources such as infrared, near-infrared, ultraviolet, microwave and/or visible light sources. Preferably, the light energy is selected to increase absorption by the shape memory polymer and reduce absorption by the surrounding tissue. Thus, damage to the tissue surrounding the shape memory polymer is reduced when the shape memory polymer is heated to change its shape. In other embodiments, the shape memory polymer comprises gas bubbles or bubble containing liquids, such as fluorocarbons, and is heated by inducing a cavitation effect in the gas/liquid when exposed to HIFU energy. In other embodiments, the shape memory polymer may be heated using electromagnetic fields and may be coated with a material that absorbs electromagnetic fields.

Certain metal alloys have shape memory qualities and respond to changes in temperature and/or exposure to magnetic fields. Exemplary shape memory alloys that respond to changes in temperature include alloys of titanium-nickel, copper-zinc-aluminum, copper-aluminum-nickel, iron-manganese-silicon, iron-nickel-aluminum, gold-cadmium, combinations of the same, and the like.

Shape memory alloys can exist in at least two distinct solid phases called martensite and austenite. In the martensite phase, the alloy is relatively soft and easily deformed, whereas in the austenite phase, the alloy is relatively stronger and less easily deformed. For example, shape memory alloys generally enter the austenite phase at a higher temperature relative to entering the martensite phase. Shape memory alloys begin transforming to the martensite phase at a martensite start temperature (Mₛ) and finish transforming to the martensite phase at a martensite finish temperature (M_{f}). Similarly, such shape memory alloys begin transforming to the austenite phase at an austenite start temperature (Aₛ) and finish transforming to the austenite phase at an austenite finish temperature (A_{f}). In general, both transformations have a hysteresis. Thus, the Mₛ temperature and the A_{f} temperature are not coincident with each other, and the M_{f} temperature and the Aₛ temperature are not coincident with each other.

In certain embodiments, the shape memory alloy is processed to form a memorized arcuate shape in the austenite phase. The shape memory alloy is then cooled below the M_{f} temperature to enter the martensite phase and deformed into a different configuration, such as substantially straight or second arcuate shape having more or less of a curve. In certain embodiments, the shape memory alloy is sufficiently malleable in the martensite phase to allow a user such as a physician to adjust the shape of the device in the martensite phase by hand to achieve a desired fit for a particular patient. After the device is positioned around or within the valve annulus, the shape of the device can be adjusted non-invasively by heating the shape memory alloy to an activation temperature (e.g., temperatures ranging from the Aₛ temperature to the A_{f} temperature).

Thereafter, when the shape memory alloy is exposed to a temperature elevation and transformed to the austenite phase, the alloy changes in shape from the deformed shape to the memorized shape. Activation temperatures at which the shape memory alloy causes the shape of the tissue shaping device to change shape can be selected for the tissue shaping device such that collateral damage is reduced or eliminated in tissue adjacent the device during the activation process. In certain embodiments, exemplary A_{f} temperatures for suitable shape memory alloys range between approximately 45° C. and approximately 50° C., and exemplary Aₛ temperatures range between approximately 42° C. and approximately 53° C. Furthermore, exemplary Mₛ temperatures range between approximately 10° C. and approximately 20° C., and exemplary M_{f} temperatures range between approximately −1° C. and approximately 15° C. The shape of the tissue shaping device can change substantially instantaneously or incrementally in small steps in order to achieve the adjustment necessary to produce the desired clinical result.

Certain shape memory alloys may further include a rhombohedral phase, having a rhombohedral start temperature (Rₛ) and a rhombohedral finish temperature (R_{f}), that exists between the austenite and martensite phases. An example of such a shape memory alloy is a NiTi alloy, which is commercially available from Memry Corporation (Bethel, Conn.). In certain embodiments, an exemplary Rₛ temperature range is between approximately 30° C. and approximately 50° C., and an exemplary R_{f} temperature range is between approximately 20° C. and approximately 35° C. One benefit of using a shape memory material having a rhombohedral phase is that in the rhomobohedral phase the shape memory material may experience a partial physical distortion, as compared to the generally rigid structure of the austenite phase and the generally deformable structure of the martensite phase.

Certain shape memory alloys exhibit a ferromagnetic shape memory effect, wherein the shape memory alloy transforms from the martensite phase to the austenite phase when exposed to a magnetic field. Thus, a tissue shaping device comprising a ferromagnetic shape memory alloy may be implanted in a first configuration having a first shape and later changed to a second configuration having a second (e.g., memorized) shape without heating the shape memory material above the Aₛ temperature. Advantageously, nearby healthy tissue is not exposed to high temperatures that could damage the tissue. Furthermore, since the ferromagnetic shape memory alloy does not need to be heated, the size and/or shape of the tissue shaping device can be adjusted more quickly and more uniformly than by heat activation.

Exemplary ferromagnetic shape memory alloys include Fe—C, Fe—Pd, Fe—Mn—Si, Co—Mn, Fe—Co—Ni—Ti, Ni—Mn—Ga, Ni₂MnGa, Co—Ni—Al, and the like. Certain of these shape memory materials may also change shape in response to changes in temperature. Thus, the shape of such materials can be adjusted by exposure to a magnetic field, by changing the temperature of the material, or both.

In certain embodiments, combinations of different shape memory materials are used. For example, tissue shaping devices according to certain embodiments comprise a combination of shape memory polymer and shape memory alloy (e.g., NiTi). In certain such embodiments, a tissue shaping device comprises a shape memory polymer body and a shape memory alloy (e.g., NiTi) disposed within the body. Such embodiments are flexible and allow the size and shape of the shape memory alloy to be further reduced without impacting fatigue properties. In addition, or in other embodiments, shape memory polymers are used with shape memory alloys to create a bi-directional (e.g., capable of expanding and contracting) tissue shaping device. Bi-directional tissue shaping devices can be created with a wide variety of shape memory material combinations having different characteristics.

In certain embodiments, the tissue shaping device includes at least one electromagnetic material configured to be activated to dynamically change the shape and/or size of the tissue shaping device. For example, the electromagnetic material, when activated, may interact with another portion of the tissue shaping device, such as a permanent magnet or other ferromagnetic material, to change the shape of the device. In one embodiment, the electromagnetic material is activated by an electromagnetic transmitter, such as a resistive coil, located outside the body of the patient.

The term “ferromagnetic” as used herein is a broad term and is used in its ordinary sense and includes, without limitation, any material that easily magnetizes, such as a material having atoms that orient their electron spins to conform to an external magnetic field. Ferromagnetic materials include permanent magnets, which can be magnetized through a variety of modes, and materials, such as metals, that are attracted to permanent magnets. Ferromagnetic materials also include electromagnetic materials that are capable of being activated by an electromagnetic transmitter, such as one located outside the heart of a patient.

Furthermore, ferromagnetic materials may include one or more polymer-bonded magnets, wherein magnetic particles are bound within a polymer matrix, such as a biocompatible polymer. The magnetic materials can comprise isotropic and/or anisotropic materials, such as for example NdFeB (Neodynium Iron Boron), SmCo (Samarium Cobalt), ferrite and/or AlNiCo (Aluminum Nickel Cobalt) particles. The biocompatible polymer can comprise, for example, polycarbonate, silicone rubber, polyurethane, silicone elastomer, a flexible or semi-rigid plastic, combinations of the same and the like.

In the following description, reference is made to the accompanying drawings, which form a part hereof, and which show, by way of illustration, specific embodiments or processes in which the invention may be practiced. Where possible, the same reference numbers are used throughout the drawings to refer to the same or like components. In some instances, numerous specific details are set forth in order to provide a thorough understanding of the present disclosure. The present disclosure, however, may be practiced without the specific details or with certain alternative equivalent components and methods to those described herein. In other instances, well-known components and methods have not been described in detail so as not to unnecessarily obscure aspects of the present disclosure.

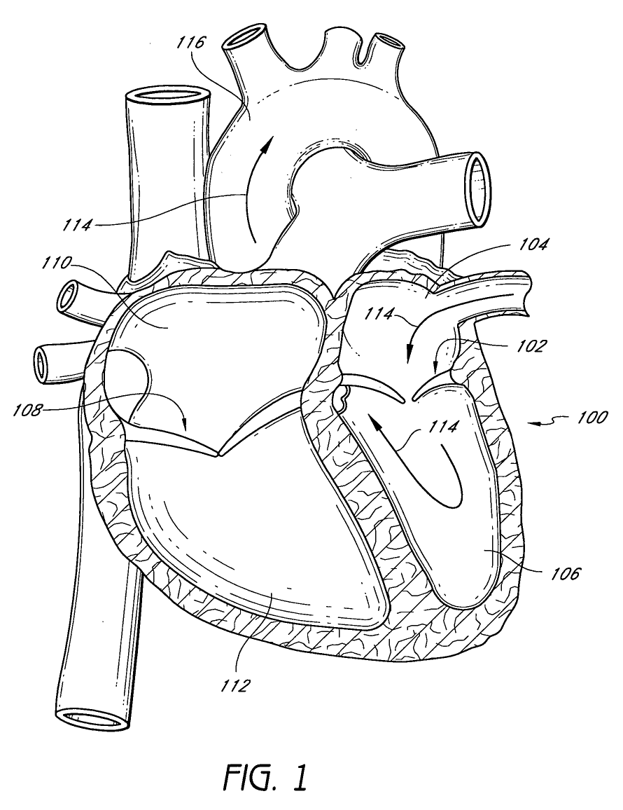

FIG. 1 is a cross-sectional view of a human heart 100, with some features of the heart 100 not shown for clarity. FIG. 1 generally illustrates the mitral (left atrioventricular) valve 102 located between the left atrium 104 and left ventricle 106, and the tricuspid valve 108 located between the right atrium 110 and the right ventricle 112. The right ventricle 112 pumps oxygen poor blood from the body to the lungs. As shown by arrows 114, blood returns from the lungs to the left atrium 104 where it is pumped through the mitral valve 102 into left ventricle 106. From the left ventricle 106, blood is pumped into the aorta to be recirculated throughout the tissues and organs of the body and returned to the right atrium 110.

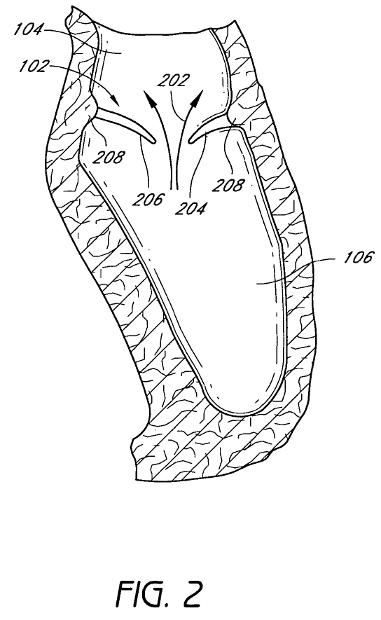

FIG. 2 is a cross-sectional view of the left ventricle 106 of the heart 100 shown in FIG. 1 illustrating regurgitation of blood back into the left atrium 104 during systole by arrows 202. The mitral valve 102 includes a first leaflet 204, a second leaflet 206 and an annulus 208. The annulus 208 may also be known as a fibrous ring. When healthy, the mitral valve annulus 208 encircles the leaflets 204, 206 and maintains their spacing to provide closure during left ventricular contraction. Regurgitation of blood from the left ventricle 106 to the left atrium 104 is due to defective closure of the leaflets 204, 206 of the mitral valve 102.

Magnetic Tissue Shaping Devices

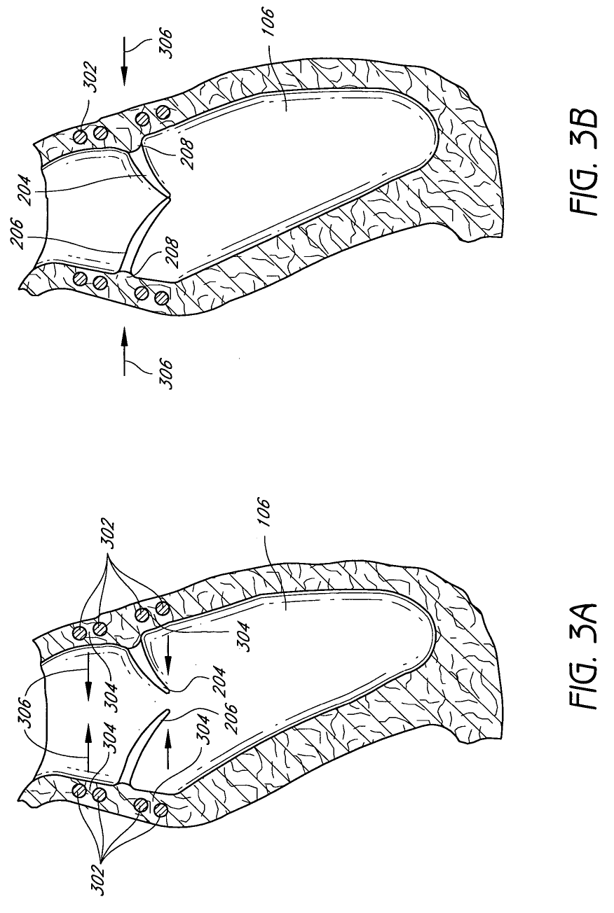

FIGS. 3A and 3B are cross-sectional views of the left ventricle 106 of the heart 100 shown in FIG. 1 illustrating a plurality of magnetic tissue shaping devices 302 implanted in myocardial tissue 304 adjacent the mitral valve leaflets 204, 206 according to certain embodiments. The placement and magnetic orientation of the tissue shaping devices 302 produce a mutually attractive force between at least two of the tissue shaping devices 302, as indicated by arrows 306. The mutually attractive force is configured to bring the myocardial tissue 304 adjacent the valve leaflets 204, 206 closer together, which, in turn, brings the valve leaflets 204, 206 closer together, as shown in FIG. 3B. Thus, the tissue shaping devices 302 are advantageously capable of reshaping at least one dimension of the mitral valve annulus 208 to improve coaptation of the valve leaflets 204, 206 during systole and reduce regurgitation caused by mitral valve insufficiency.

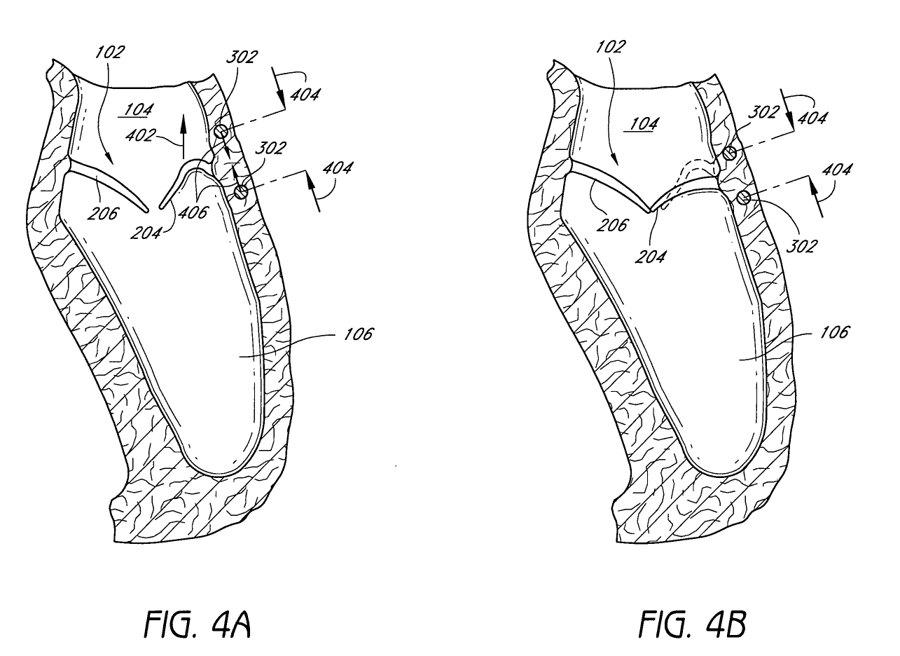

FIGS. 4A and 4B are cross-sectional views of the left ventricle 106 of the heart 100 shown in FIG. 1 illustrating two mutually attractive magnetic tissue shaping devices 302 implanted on opposite sides of the plane of the mitral valve 102 adjacent the leaflet 204. As illustrated in FIG. 4A, the leaflet 204 is in a prolapsed condition, as indicated by arrow 402, in which the leaflet 204 protrudes into the left atrium 104 and does not achieve sufficient coaptation with the other leaflet 206. The tissue shaping devices 302 are initially separated upon deployment by a distance which is indicated by the dashed lines and arrows 404. Once the tissue shaping devices 302 are free of the deployment device or catheter, the mutual attraction will pull the two tissue shaping devices 302 towards one another, as indicated by arrows 406.

As illustrated in FIG. 4B, the attraction between the tissue shaping devices 302 remodels the tissue adjacent the tissue shaping devices 302 such that the anchor point of the leaflet 204 adjacent the tissue shaping devices 302 is displaced. The downward displacement of the anchor point of the leaflet 204, as compared to the original position of the anchor point shown in dashed lines, is due to the decrease in the distance between the two tissue shaping devices 302. The downward shift of the anchor point of the valve leaflet 204 reduces the prolapse of the valve leaflet 204 and improves valve closure and function.

Referring to FIGS. 3A-4B, the tissue shaping devices 302 may have any suitable configuration provided they produce a strong magnetic field relative to their size and are suitably biocompatible for implantation in the human body. In certain embodiments, one or more of the tissue shaping devices 302 comprise a magnetic material. The tissue shaping devices 302, according to certain embodiments, are disc shaped magnets having aligned poles for attraction and opposing poles for repulsion depending on the desired effect. In certain such embodiments, one or more of the tissue shaping devices 302 has a diameter and/or thickness in a range between approximately 0.25 mm and approximately 0.5 mm, which facilitates placement and/or removal of the tissue shaping devices 302 from the myocardial tissue 304. In certain other embodiments, one or more of the tissue shaping devices 302 may be in the shape of a rod, a sphere, a cylinder, a cube or the like. In certain such embodiments, one or more of the tissue shaping devices 302 includes a magnetic rod having a length in a range between approximately 3.0 mm and approximately 8.0 mm and a transverse dimension or diameter in a range between approximately 0.25 mm and approximately 0.5 mm.

The shape or shapes selected for the tissue shaping devices 302 may depend, at least in part, on factors such as the desired field to be produced, the resulting mutual force or forces between the tissue shaping devices 302, combinations of the foregoing, or the like. Any suitable number of tissue shaping devices 302 may be used for a particular procedure. For some procedures, the number of tissue shaping devices 302 implanted may be in a range between approximately two tissue shaping devices 302 to approximately twenty tissue shaping devices 302. In certain other embodiments, more than twenty tissue shaping devices 302 may be used.

In certain embodiments, the tissue shaping devices 302 advantageously comprise a ferromagnetic material. In certain preferred embodiments, at least one of the tissue shaping devices 302 includes one or more rare-earth elements or rare-earth alloys, such as alloys of NdFeB (Neudynium Iron Boron), SmCo (Samarium Cobolt), AlNiCo (Aluminum Nickel Cobalt), combinations of the foregoing, or the like.

In certain embodiments, one or more of the tissue shaping devices 302 can produce a force in a range between approximately 0.2 lbf and approximately 0.5 lbf with a magnetic field in a range between approximately 300 Gauss and approximately 3000 Gauss. In certain embodiments, the outside surface of the tissue shaping devices 302 is coated with a thin coating of biocompatible polymeric material such as polyurethane, polytetrafluoroethylene (PTFE), fluorinated ethylene propylene (FEP) or polyether ether kythane (PEEK®). An outer layer of stainless steel, such as 3166 stainless steel, or other suitable biocompatible alloy, may also be used.

Although disclosed with reference to particular embodiments, the tissue shaping devices 302 may include a wide variety of alternative forms and/or shapes. For example, in certain embodiments, the tissue shaping devices 302 include at least one permanent magnet, such as a rare-earth alloy, and at least one generally unmagnetized ferromagnetic portion that responds to the magnetic field emanated by the permanent magnet(s). In certain other embodiments, the tissue shaping devices 302 include at least one electromagnet. In such an embodiment, an electromagnetic transmitter, such as a resistive coil, may be used to activate the electromagnet(s). The transmitter may advantageously be located outside the heart 100 and may be usable to non-invasively magnetize one or more of the tissue shaping devices 302 after the tissue shaping devices 302 have been positioned within the myocardial tissue 304.

In certain other embodiments, the tissue shaping devices 302 may comprise at least one magnetic structure including a magnet comprising a hard ferromagnetic material and a magnetic flux shield comprising a soft ferromagnetic material overlaying at least a portion of the magnet. The flux shield may be used to focus and enhance the magnetic field of the magnet in a direction that the shield does not overlay (e.g., in the direction of another magnet).

In general, the tissue shaping devices 302 interact with each other to cause a change in the shape of the myocardial tissue 304, which, as discussed above, effects a change in the shape of the mitral valve annulus 208. As discussed below, in certain embodiments, the tissue shaping devices 302 are implanted in the mitral valve annulus 208 and interact with each other to cause a change directly to the mitral valve annulus 208. Regardless of the location of the tissue shaping devices 302, in certain embodiments, the interaction is a magnetic interaction that causes attraction (e.g., between poles of different polarity) and/or repulsion (e.g., between poles of like polarity) between the tissue shaping devices 302.

Shape Memory Tissue Shaping Devices

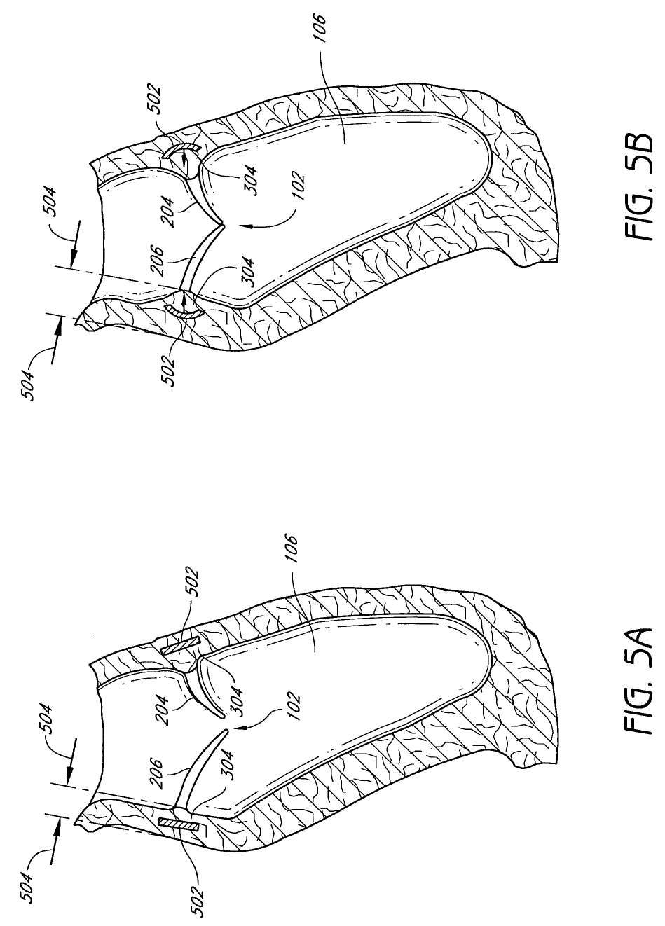

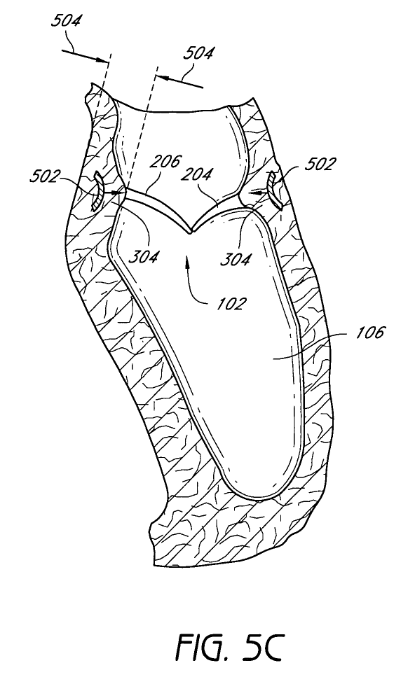

FIGS. 5A-5C are cross sectional views of the left ventricle 106 of the heart 100 shown in FIG. 1 illustrating two tissue shaping devices 502 comprising a shape memory material (such as the shape memory materials discussed above) deployed in heart tissue 304 adjacent the leaflets 204, 206 of the mitral valve 102 according to certain embodiments. The heart tissue 304 where the tissue shaping devices 502 may be employed include, for example, myocardium, the interventricular septum of the heart 100, the left and/or right fibrous trigone, or a wall of the left atrium 104 or right atrium 110. In FIG. 5A, the tissue shaping devices 502 are in a first configuration wherein the shape memory materials are in an inactivated (e.g., martensite) state. In the first configuration, the tissue shaping devices 502 are substantially straight. In certain embodiments, this substantially straight configuration is selected to advantageously facilitate placement of the tissue shaping devices 502 into the myocardial tissue 304 with a longitudinal axis of the tissue shaping devices 502 being substantially perpendicular to the plane of the mitral valve 102. In certain other embodiments, the tissue shaping devices 502 in the first configuration have a slightly arcuate shape.

After the tissue shaping devices 502 have been implanted in the myocardial tissue 304, a suitable energy source, such as one or more of the energy sources discussed above, is used to activate the tissue shaping devices 502. As shown in FIGS. 5B and 5C, in the activated (e.g., austenite) state, the tissue shaping devices 502 transform to a second configuration having an arcuate shape with a greater curvature than the substantially straight or slightly arcuate shape of the first configuration. When activated, the tissue shaping devices 502 reshape the myocardial tissue 304 in the region adjacent the base of the mitral valve leaflets 204, 206, as indicated by the dashed lines and distance between arrows 504. As shown, the distance indicated by arrows 504 is wider in FIGS. 5B and 5C after activation than in FIG. 5A prior to activation. Thus, the deformation of the tissue shaping devices 504 advantageously forces the inward tips of the leaflets 204, 206 toward one another so as to form a better seal and valve function.

In certain embodiments, the tissue shaping devices 502 cause a pressure or force in a range between approximately 2.22 newtons (0.5 pound-force) and approximately 13.34 newtons (3.0 pound-force) of displacement on the myocardial tissue 304 to change at least one dimension of the mitral valve 102. Such pressure may cause the leaflets 204, 206 to move a distance in a range between approximately 5.0 mm and 15.0 mm toward one another. In certain embodiments, the tissue shaping devices 502 are configured to push the leaflets toward one another a distance in a range between approximately 2.0 mm and approximately 30.0 mm.

As shown in FIG. 5B, in certain embodiments, the tissue shaping devices 502 are implanted in the myocardial tissue 304 such that the ends of the tissue shaping devices 502 push in the general direction of the mitral valve 208 when activated. In particular, the tissue shaping devices 502 dynamically adjust such that a concave portion or side of the tissue shaping devices 502 pushes the leaflets 204, 206 towards one another to facilitate greater coaptation. In other embodiments, as shown in FIG. 5C, the tissue shaping devices 502 are implanted in the myocardial tissue 304 such that a convex portion or side of the tissue shaping devices 502 bows toward the mitral valve 208 when activated, which causes movement of the leaflets 204, 206 toward one another to facilitate greater coaptation.

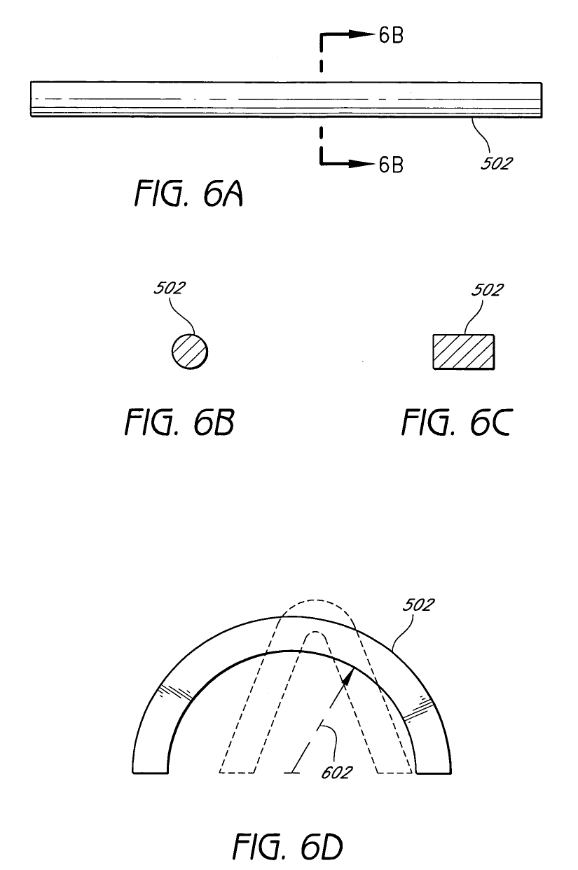

FIGS. 6A-6D schematically illustrate exemplary embodiments of the tissue shaping device 502 capable of transforming from a first configuration to a second configuration according to certain embodiments. The tissue shaping device 502 comprises a shape memory material, such as one or more of the shape memory materials discussed above. In FIG. 6A, the tissue shaping device 502 is shown in the first configuration wherein the shape memory material has not been activated (e.g., the shape memory material is in the martensite state). In certain embodiments, the tissue shaping device 502 has an elongate body having its longest length in a range between approximately 3.0 mm and approximately 8 mm. In an exemplary embodiment, the longest length of the tissue shaping device 502 is approximately 6 mm. In other embodiments, the longest length of the tissue shaping device 502 is in a range between approximately 8 mm and approximately 15 mm.

As shown in FIG. 6B, in certain embodiments, the tissue shaping device 502 has a round transverse cross section. In certain such embodiments, the tissue shaping device 502 has a diameter or transverse dimension in a range between approximately 0.005 inches and approximately 0.020 inches. An artisan will recognize from the disclosure herein that the tissue shaping device 502 can have other cross-sectional shape including, for example, oval, square, rectangular, or any other polygonal shape. For example, FIG. 6C shows a transverse cross section of an alternative embodiment of a shape memory member 502 having a rectangular cross section.

In FIG. 6D, the tissue shaping device 502 is shown in the second configuration (represented by solid lines) wherein the shape memory material is in an activated state (e.g., austenite state). In the second configuration, the tissue shaping device 502 has an arcuate shape with a radius of curvature indicated by arrow 602. In certain embodiments, the radius of curvature in the activated state is in a range between approximately 0.10 inches and approximately 0.30 inches. In addition or in other embodiments, the tissue shaping device 502 is adjustable to a third configuration (represented by dashed lines in FIG. 6D). In the third configuration, the ends of the tissue shaping device 502 are closer together than in the second configuration. In such embodiments, the third configuration is advantageously usable to cause an increased pressure on the myocardial tissue 304 and a corresponding pressure on the mitral valve annulus 208. Thus, the tissue shaping device 502 can be further adjusted as needed to provide further reinforcement and increased leaflet coaptation.

In certain other embodiments, the ends of the tissue shaping device 502 move further apart as the tissue shaping device 502 transitions from the second configuration to the third configuration. In such embodiments, the tissue shaping device 502 applies less pressure to the myocardial tissue 304 and mitral valve annulus 208 in the third configuration than in the second configuration. Advantageously, this allows the size of the mitral valve annulus 208 to be reshaped as an enlarged heart returns to its normal size.

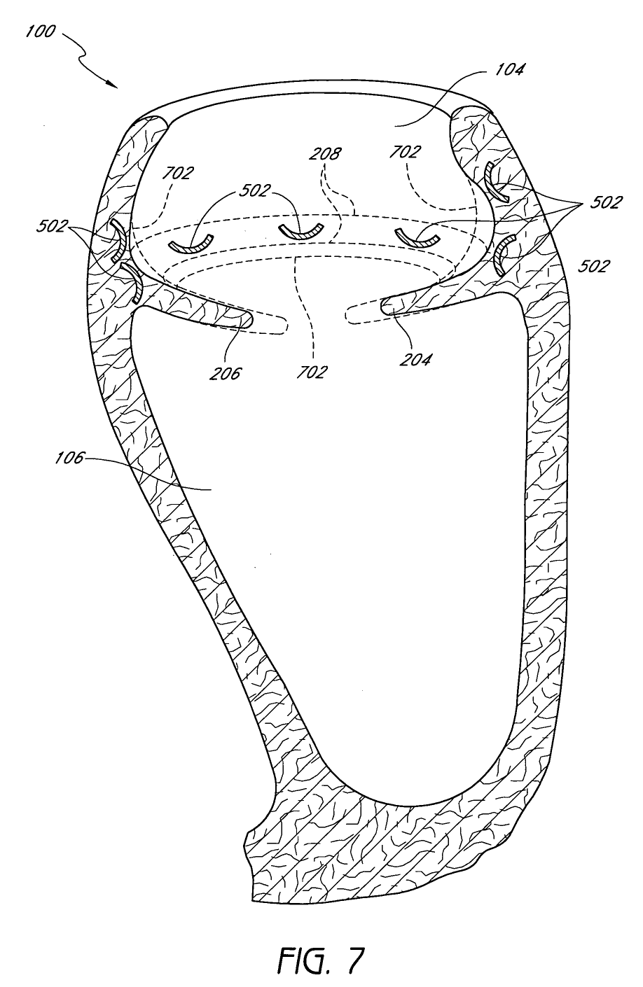

An artisan will recognize from the disclosure herein that one tissue shaping device 502, two tissue shaping devices (as shown in FIGS. 5A-5C), or more than two tissue shaping devices 502 can be used to achieve desired reshaping of the mitral valve annulus 208. For example, FIG. 7 is a cross-sectional view of the left ventricle 106 of the heart 100 illustrating a plurality of tissue shaping devices 502 comprising a shape memory material implanted within the mitral valve annulus 208 (illustrated with a first set of dashed lines). As shown in FIG. 7, in certain such embodiments, the tissue shaping devices 502 are implanted in a first configuration having an arcuate shape. Upon activation, the tissue shaping devices 502 transform to a second configuration having a greater arcuate shape than the first configuration. The leaflets 204, 206 are closer together when the tissue shaping devices 502 are in the second configuration (e.g., the leaflets 204, 206 are shown in dashed lines) than when the tissue shaping devices 502 are in the first configuration (e.g., the leaflets 204, 206 are shown in solid lines). As illustrated by dashed lines 702, the mitral valve annulus 208 also has a smaller circumference when the tissue shaping devices 502 are activated.

Advantageously, in certain embodiments, the tissue shaping devices 502 can be selectively activated post-implantation so as to reshape portions of the mitral valve annulus 208. For example, in certain such embodiments, one or more of the tissue shaping devices 502 are configured to be activated at a first temperature and one or more other tissue shaping devices 502 are configured to be activated at a second temperature. In addition or in other embodiments, one or more of the tissue shaping devices 502 are configured to be activated in response to a first electromagnetic wave having a first frequency and one or more other tissue shaping devices 502 are configured to be activated in response to a second electromagnetic wave having a second frequency. Thus, the mitral valve annulus 208 can be selectively reshaped in one or more dimensions at a time. By selecting one or more of the tissue shaping devices 502 to activate at a time, the mitral valve annulus 208 can be gradually resized in steps until the desired coaptation between the leaflets 204, 206 is achieved.

In certain embodiments, the tissue shaping devices 502 are configured to exert different pressures on their respective locations in or around the mitral valve annulus 208. In addition or in other embodiments, alternative configurations, shapes, sizes and the like may be used with at least one of the plurality of tissue shaping devices 502. In yet other embodiments, additional or fewer tissue shaping devices 502 may be used to achieve a certain therapeutic outcome with respect to the mitral valve annulus 208. In yet other embodiments, two or more tissue shaping devices 502 may be positioned side-by-side in a parallel configuration to effect corresponding changes in the mitral valve annulus 208. In yet other embodiments, the tissue shaping devices 502 may be of different lengths, different shapes, or otherwise modified to provide for variable forces upon the mitral valve annulus 208 and leaflets 204, 206.

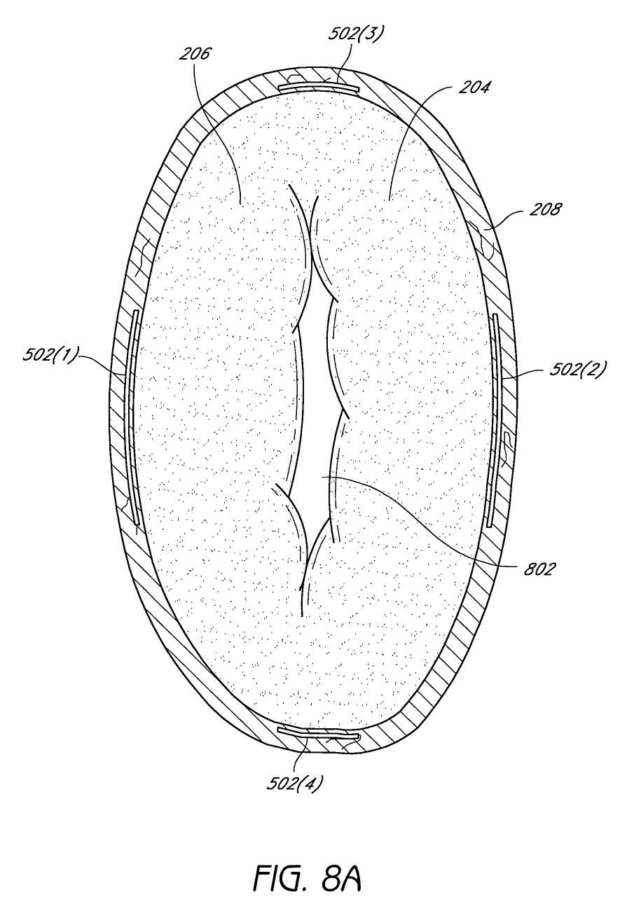

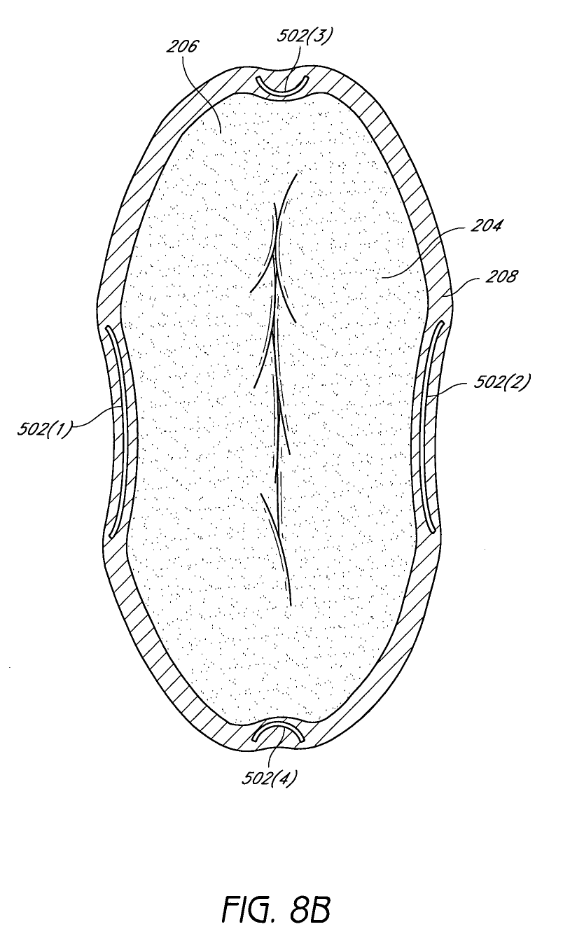

FIGS. 8A and 8B illustrate top schematic views of four tissue shaping devices 502(1)-502(4) implanted in the mitral valve annulus 208 according to an exemplary embodiment. A first tissue shaping device 502(1) and a second tissue shaping device 502(2) are implanted on the anterior and posterior sides of the mitral valve annulus 208. A third tissue shaping device 502(3) and a fourth tissue shaping device 502(4) are implanted in the top and bottom of the mitral valve annulus 208, respectively, approximately half-way between the first tissue shaping device 502(1) and the second tissue shaping device 502(2). In certain embodiments, the four tissue shaping devices 502(1)-502(4) are activated one at a time until a desired therapeutic effect is achieved. FIG. 8A illustrates the tissue shaping devices 502(1)-502(4) before activation and FIG. 8B illustrates the tissue shaping devices 502(1)-502(4) after activation.

For example, the first tissue shaping device 502(1) is activated by applying energy thereto, as discussed herein, so as to raise the temperature of its shape memory material to a first activation temperature. Once the first tissue shaping device 502(1) is activated, it pushes the leaflets 204, 206 together in the anterior/posterior direction. Imaging is then used, according to certain embodiments, to determine if sufficient coaptation between the leaflets 204, 206 has been achieved so as to close a gap 802 between the leaflets 204, 206 and reduce regurgitation below a desired level.

If sufficient coaptation has not been achieved, energy can again be applied so as to raise the temperature of the second tissue shaping device 502(2) above a second activation temperature to activate its shape memory material. In certain such embodiments, the second activation temperature is higher than the first activation temperature. Once the second tissue shaping device 502(2) is activated, it pushes the leaflets 204, 206 further together in the anterior/posterior direction. The regurgitation can again be measured to determine if sufficient coaptation has been achieved. Thus, the first tissue shaping device 502(1) and the second tissue shaping device 502(2) can be used to reshape the mitral valve annulus 208 in the anterior/posterior direction and sufficiently reduce regurgitation.

If further reshaping is required, the third tissue shaping device 502(3) and the fourth tissue shaping device 502(4) can be activated by successively heating them to a third activation temperature and a fourth activation temperature, respectively. In certain such embodiments, the third activation temperature is higher than the second activation temperature and the fourth activation temperature is higher than the third activation temperature. In certain other embodiments, two or more of the tissue shaping devices 502(1)-502(4) are activated at the same time. For example, the third tissue shaping device 502(3) and the fourth tissue shaping device 502(4) may both be activated upon reaching the third activation temperature. In certain other embodiments, one or more of the tissue shaping devices 502(1)-502(4) may be activated using different forms of energy and/or without substantial heating. For example, at least one of the first tissue shaping device 502(1) and the second tissue shaping device 502(2) may be activated in response to being heated with focused ultrasound energy and at least one of the third tissue shaping device 502(3) and the fourth tissue shaping device 502(4) may comprise a ferromagnetic shape memory alloy configured to be activated without substantial heating when exposed to a magnetic field.

Although described with reference to particular embodiments, the tissue shaping device 502 may take on other forms or configurations that are suitable for reshaping the mitral valve annulus 208. For example, embodiments of the tissue shaping device 502 may transform between only two configurations (e.g., at the austenite and martensite phases), or the tissue shaping device 502 may experience transformations between more than three configurations. Furthermore, other embodiments of the tissue shaping device 502 may experience changes in dimensions other than, or in combination with, a bending of the tissue shaping device so as to move its opposite ends closer together. For example, only a select segment of the tissue shaping device 502 may undergo a shape transformation, such as, for example, a segment consisting essentially of a shape memory material.

Deformation of the tissue shaping device 502 from at least the first configuration to the second configuration may be performed in several ways. In certain embodiments, the tissue shaping device 502 comprises a shape memory material that is responsive to changes in temperature and/or exposure to a magnetic field. With reference to FIGS. 6A and 6D, in certain embodiments, the tissue shaping device 502 includes at least one shape memory portion usable to adjust the tissue shaping device 502 from the first configuration to the second configuration. For example, the first configuration may correspond to when the shape memory portion is in the martensite phase, and the second configuration may correspond to when the shape memory portion is in the austenite phase. In other embodiments, the second configuration may correspond to when the shape memory material is in the rhombohedral phase, and the third configuration may correspond to when the shape memory material is in the austenite phase.

As discussed above, the shape memory material may include shape memory polymers (e.g., polylactic acid (PLA), polyglycolic acid (PGA)) and/or shape memory alloys (e.g., nickel-titanium) including, for example, ferromagnetic shape memory alloys (e.g., Fe—C, Fe—Pd, Fe—Mn—Si, Co—Mn, Fe—Co—Ni—Ti, Ni—Mn—Ga, Ni₂MnGa, Co—Ni—Al). In certain such embodiments, the tissue shaping device 502 is adjusted in vivo by applying an energy source such as, but not limited to, radio frequency energy, X-ray energy, microwave energy, acoustic energy such as HIFU energy, light energy, electric field energy, magnetic field energy, combinations of the same or the like.

Preferably, the energy source is applied in a non-invasive manner from outside the body of the patient, as is described in more detail herein. For example, a magnetic field and/or RF pulses can be applied to the tissue shaping device 502 within a patient's heart with an apparatus external to the patient's heart and/or unattached to the tissue shaping device 502. Such magnetic fields and/or RF pulses are commonly used for magnetic resonance imaging (MRI). However, in other embodiments, the energy source may be applied surgically, such as by inserting a catheter into the body and applying energy through the catheter.

In certain embodiments, the tissue shaping device 502 is selectively heated using short pulses of energy having an on and an off period between each cycle. The energy pulses provide segmental heating which allows segmental adjustment of portions of the tissue shaping device 502 without adjusting the entire implant.

In certain embodiments, the tissue shaping device 502 comprises a shape memory material that responds to a change in temperature that differs from a nominal ambient temperature, such as the nominal body temperature of 37° C. for humans. For example, the tissue shaping device 502 may be configured to respond by starting to contract upon heating of the tissue shaping device 502 above the Aₛ temperature of the shape memory material.

The activation temperatures (e.g., temperatures ranging from the Aₛ temperature to the A_{f} temperature) at which the tissue shaping device 502 contracts (e.g., increased vertical dimension) may be selected for the tissue shaping device 502 such that collateral damage is reduced or eliminated in tissue adjacent the tissue shaping device 502 during the activation process. Exemplary A_{f} temperatures for the shape memory material of the tissue shaping device 502 at which substantially maximum contraction occurs are in a range between approximately 38° C. and approximately 75° C. For some embodiments that include shape memory polymers for the tissue shaping device 502, activation temperatures at which the glass transition of the material or substantially maximum contraction occur range between approximately 38° C. and approximately 60° C. In other such embodiments, the activation temperature is in a range between approximately 45° C. and approximately 50° C.

In certain embodiments, the tissue shaping device 502 is shape set in the austenite phase to a remembered configuration during the manufacturing of the tissue shaping device 502 such that the remembered configuration is arcuately shaped and has a relatively long vertical dimension. After cooling the tissue shaping device 502 below the M_{f} temperature, the tissue shaping device 502 is manually deformed into a shape having a shorter vertical dimension. In certain such embodiments, the tissue shaping device 502 is sufficiently malleable in the martensite phase to allow a user such as a physician to adjust the shape by hand to achieve a desired size for implantation. In certain embodiments, the starting shape of the tissue shaping device 502 is selected to improve leaflet coaptation and reduce regurgitation in the mitral valve 102.

For embodiments of the tissue shaping device 502 made from a continuous piece of shape memory alloy (e.g., NiTi alloy) or shape memory polymer, the tissue shaping device 502 can be activated by the surgical and/or non-invasive application of heating energy by the methods discussed herein. For embodiments of the tissue shaping device 502 made from a continuous piece of ferromagnetic shape memory alloy, the tissue shaping device 502 can be activated by the non-invasive application of a suitable magnetic field.

Alternatively, the tissue shaping device 502 may comprise two or more sections or zones of shape memory material having different temperature response curves. The shape memory response zones may be configured in order to achieve a desired configuration of the tissue shaping device 502 when in a contracted state, either fully contracted or partially contracted.

In certain embodiments, the shape memory portion of the tissue shaping device 502 extends more than half the length of the tissue shaping device 502. In embodiments of the invention having multiple shape memory portions, the total length of the shape memory portions may exceed half the length of the tissue shaping device 502 while one or more of the multiple portions may have an individual length of less than half the length of the tissue shaping device 502.

The shape modification process of the tissue shaping device 502, either non-invasively or through a catheter, can be carried out all at once or incrementally in order to produce the desired clinical result. If heating energy is applied such that the temperature of the tissue shaping device 502 does not reach the A_{f} temperature for substantially maximum transition contraction, partial shape memory transformation and contraction may occur.

After implantation, the tissue shaping device 502 is preferably activated non-invasively by the application of energy to the patient's body to heat the tissue shaping device 502. In certain embodiments, an MRI device is used as discussed above to heat the tissue shaping device 502, which then causes the shape memory material of the tissue shaping device 502 to transform to the austenite phase and its associated (contracted) configuration. Thus, the shape of the tissue shaping device 502 is changed in vivo without the need for surgical intervention. Standard techniques for focusing the magnetic field from the MRI device onto the tissue shaping device 502 may be used. For example, a conductive coil can be wrapped around the patient in an area corresponding to the tissue shaping device 502. In other embodiments, the shape memory material is activated by exposing it to other sources of energy, as discussed above.

The shape change of the tissue shaping device 502 can be assessed or monitored using MRI imaging, ultrasound imaging, computed tomography (CT) scan, X-ray or the like. If magnetic energy is being used to activate contraction of the tissue shaping device 502, for example, MRI imaging techniques can be used that produce a field strength that is lower than that required for activation of the tissue shaping device 502.

In addition to the foregoing, embodiments of the tissue shaping devices described herein may include at least one passive fixation mechanism for securing the tissue shaping devices to the myocardial tissue 304. Such passive fixation mechanisms allow for the tissue shaping device to be temporarily or permanently attached to a surface on or near the mitral valve annulus 208 rather than within the myocardial tissue, as illustrated in FIGS. 5A-5C, 7 and 8A-8B.

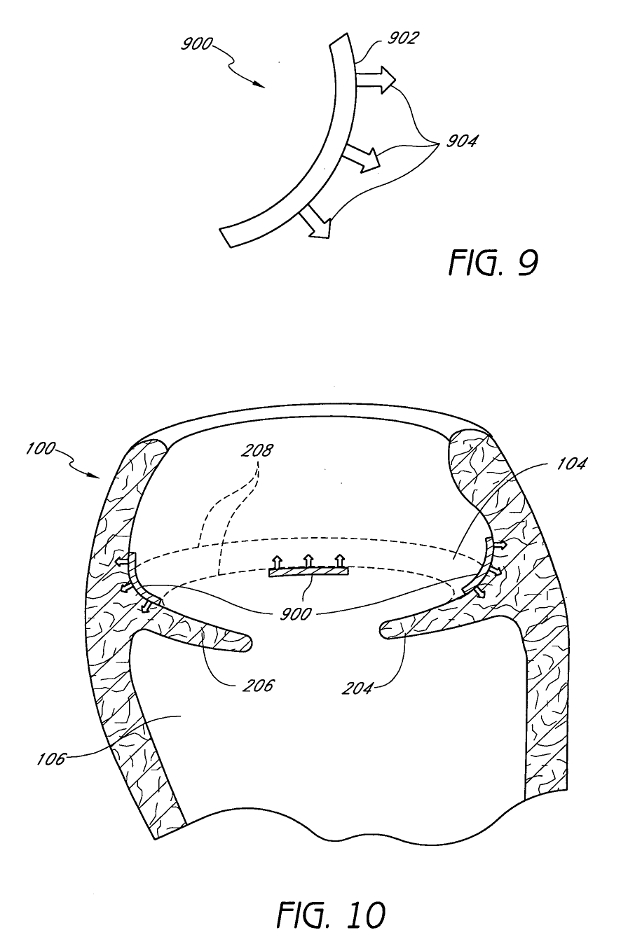

For example, FIG. 9 is a schematic diagram illustrating a tissue shaping device 900 including a shape memory body member 902 defining one or more fixation anchors 904 on a surface thereof. In certain embodiments, the body member is substantially similar to the tissue shaping device 502 described above. The anchors 904 may be connected to, or incorporated in, the outer surface of the tissue shaping device 900. The anchors 904 are configured to penetrate the surface of the myocardial tissue 304 so as to be securely attached on or near the mitral valve annulus 208. In certain embodiments, the anchors 904 are barbed to prevent or reduce the likelihood of detaching from the surface of the myocardial tissue 304. In certain embodiments, the anchors 904 advantageously comprise a flexible material, such as, for example, silicone or polyurethane. In other embodiments, the anchors 904 are advantageously constructed of a braided material, such as, for example, stainless steel, nylon or any other suitable combination of metals and/or polymers.

In other embodiments, other types of passive fixation mechanisms may be used. For example, the tissue shaping device 900 may include bristle-like projections, anchor pads, spikes, helical or round protrusions, combinations of the same, or the like. In addition or in other embodiments, multiple types of passive fixation mechanisms may be used with the same tissue shaping device 900. Other types of fixation mechanisms usable with embodiments of the present invention also include active fixation mechanisms, such as, for example, screw-in mechanisms or anchors comprising shape memory material.

FIG. 10 is a cross sectional view of the left ventricle 106 of the heart 100 illustrating a plurality of tissue shaping devices 900 implanted on and/or adjacent to the surface of the mitral valve annulus 208 (illustrated with a first set of dashed lines). As shown, the anchors 904 are securely embedded within the myocardial tissue 304. In certain such embodiments, the tissue shaping devices 900 are implanted in a first configuration having an arcuate shape. Upon activation, the tissue shaping devices 502 transform to a second configuration having a greater arcuate shape than the first configuration. Thus, the tissue shaping devices are configured to reshape the mitral valve annulus 208 so as to increase coaptation of the leaflets and reduce regurgitation, as discussed herein.

The tissue shaping devices 502, 900 may have different shapes or forms than the generally rod-shaped device depicted, for example, in FIGS. 6A and 6B. For example, the tissue shaping device 502 may comprise a helical shape, an arcuate shape, an S-shape, a ribbon-like shape, a curvilinear shape, a braided-wire, multiple wires, combinations of the same or the like.

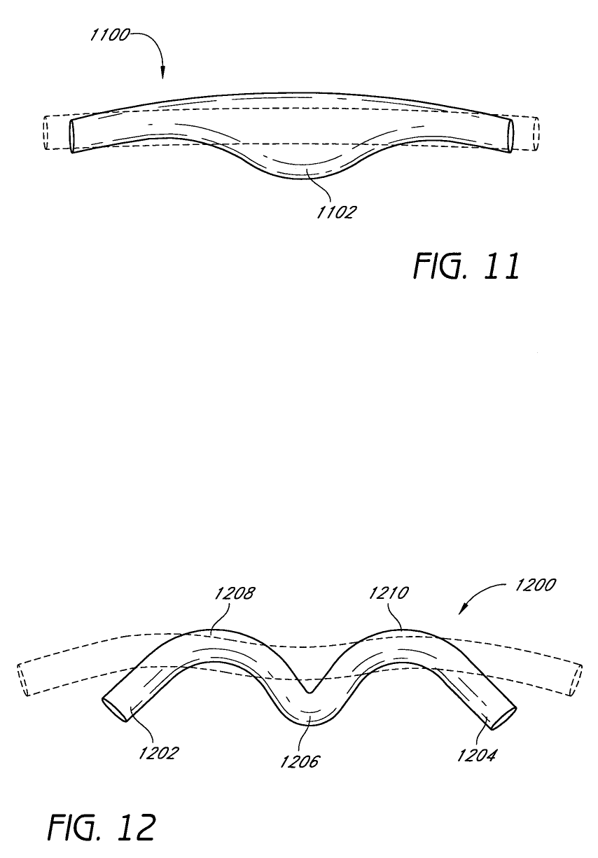

FIG. 11 schematically illustrates an exemplary embodiment of a tissue shaping device 1100 that is dynamically adjustable to effect changes in at least one dimension of the mitral valve annulus 208. The tissue shaping device 1100 has a generally uniform rod shape in a first configuration, as shown by the dashed lines. In a second configuration, the tissue shaping device 1100 forms a protrusion 1102 near the center of the length of the tissue shaping device 1100. In certain embodiments, the protrusion 1102 advantageously pushes against the myocardial tissue 304 to reshape the mitral valve annulus 208, thereby causing movement of the leaflets 204, 206 toward one another to facilitate greater coaptation.

Although disclosed with reference to particular embodiments, the tissue shaping device 1100 may take on alternative forms and/or shapes during dynamic adjustments between multiple configurations. For example, the tissue shaping device 1100 may include multiple protrusions and/or may take on an arcuate shape in the second configuration.

FIG. 12 schematically illustrates another exemplary embodiment of a tissue shaping device 1200 that is dynamically adjustable to effect changes in the shape of the mitral valve annulus 208. In particular, the tissue shaping device 1200 is adjustable between at least a first configuration (depicted as dashed lines) and a second configuration (depicted as solid lines). In the first configuration, the tissue shaping device 1200 includes a more elongated or extended shape, which advantageously facilitates deployment of the tissue shaping device 1200 within the myocardial tissue 304. In the second configuration, the tissue shaping device 1200 contracts to a wider (e.g., a longer vertical dimension) and less elongated shape.

As shown, the tissue shaping device 1200 has a curvilinear shape including ends 1202, 1204, a center protrusion 1206 and side protrusions 1208, 1210. As the tissue shaping device 1200 contracts from the first configuration to the second configuration, the center protrusion 1206 presses against the myocardial tissue 302 proximate the mitral valve annulus 208. In particular, the deformation of the tissue shaping device 1200 advantageously moves one of the leaflets 204, 204 toward the other, as discussed herein, to facilitate greater coaptation. In addition or in other embodiments, the tissue shaping device 1200 may be situated such that the side protrusions 1208, 1210 cause a change in at least one dimension of the mitral valve annulus 116.

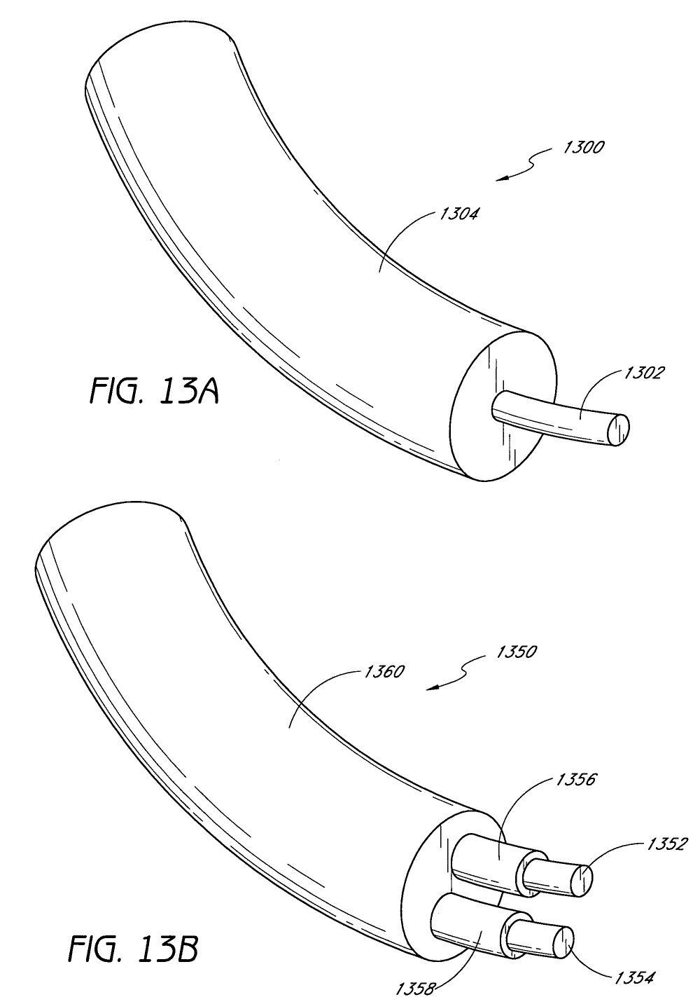

FIG. 13A is an enlarged perspective view of a portion of a tissue shaping device 1300 according to certain embodiments. The illustrated tissue shaping device 1300 includes a wire 1302 and a flexible material 1304. For illustrative purposes, portions of the flexible material 1304 are not shown so as to expose the wire 1302.

The term “wire” as use herein is a broad term having its normal and customary meaning and includes, without limitation, mesh, flat, round, band-shaped, and rod-shaped members. In certain embodiments, the wire 1302 has a diameter between approximately 0.0254 mm and approximately 0.254 mm.

In certain embodiments, the wire 1302 comprises a shape memory material. Suitable shape memory materials include shape memory polymers or shape memory alloys. For example, in certain embodiments, the wire 1302 comprises a NiTi alloy configured to transition to its austenite phase when heated and transform to a memorized shape, as discussed above. In certain such embodiments, the wire 1302 is configured to contract to an arcuate shape when transitioning to the austenite phase. In certain such embodiments, the austenite start temperature Aₛ is in a range between approximately 33° C. and approximately 43° C., the austenite finish temperature A_{f} is in a range between approximately 45° C. and approximately 55° C., the martensite start temperature Mₛ is less than approximately 30° C., and the martensite finish temperature M_{f} is greater than approximately 20° C. In other embodiments, the austenite finish temperature A_{f} is in a range between approximately 48.75° C. and approximately 51.25° C.

In certain embodiments, the shape memory material of the wire 1302 may be cooled to change shape. Certain shape memory alloys, such as NiTi or the like, respond to the application of a temperature below the nominal ambient temperature. After heating of the wire 1302 has taken place, the wire 1302 is cooled below the Mₛ temperature to start expanding the tissue shaping device 1300. The wire 1302 can also be cooled below the M_{f} temperature to finish the transformation to the martensite phase and reverse the contraction cycle.

As discussed above, certain polymers also exhibit a two-way shape memory effect and can be used in the wire 1302 to both expand and contract the tissue shaping device 1300 through heating and cooling processes. Cooling can be achieved, for example, by inserting a cool liquid onto or into the tissue shaping device 1300 through a catheter, or by cycling a cool liquid or gas through a catheter placed near the tissue shaping device 1300. Exemplary temperatures for a NiTi embodiment for cooling and reversing a contraction cycle range between approximately 20° C. and approximately 30° C.

In certain embodiments, the wire 1302 comprises an energy absorption enhancement material (not shown), which includes any material or compound that selectively absorbs and converts a non-invasive heating energy to heat, which is then transferred by thermal conduction to the wire 1302. The energy absorption enhancement material allows the tissue shaping device 1300 to be actuated and adjusted by the non-invasive application of lower levels of energy and also allows for the use of non-conducting materials, such as shape memory polymers, for the wire 1302. In certain embodiments, magnetic flux ranging between approximately 2.5 Tesla and approximately 3.0 Tesla may be used for activation. By allowing the use of lower energy levels, the energy absorption enhancement material also reduces thermal damage to nearby tissue. In addition or in other embodiments, the energy absorption enhancement material is radiopaque. Suitable energy absorption enhancement materials are discussed in more detail above.

In certain embodiments, the energy absorption enhancement material is located within the wire 1302 or may be coated on the outside of the wire 1302 to enhance energy absorption. It may also be desirable for the energy absorption enhancement material, a carrier material surrounding the energy absorption enhancement material, or both to be thermally conductive. Thus, thermal energy from the energy absorption enhancement material is transferred to the wire 1302.

In yet other embodiments, the wire 1302 comprises a ferromagnetic shape memory material, as discussed above. In such embodiments, the shape of the wire 1302 can be changed by exposing the tissue shaping device 1300 and wire 1302 to a magnetic field. When using a magnetic field to adjust the tissue shaping device 1300, nearby healthy tissue is not exposed to high temperatures that could damage the tissue. Furthermore, since the shape memory material does not need to be heated, the shape and/or size of the tissue shaping device 900 is capable of being adjusted more quickly and more uniformly than by heat activation.

With continued reference to FIG. 13A, the illustrated wire 1302 is substantially enclosed in the flexible material 1304. In certain embodiments, the flexible material 1304 advantageously comprises a biocompatible material, such as for example, silicone rubber. In other embodiments, the flexible material 1304 comprises woven polyester cloth, Dacron@, woven velour, polyurethane, polytetrafluoroethylene (PTFE), heparin-coated fabric, combinations of the same or the like. In yet other embodiments, the flexible material 1304 comprises a biological material, such as for example, bovine or equine pericardium, homograft, patient graft, or cell-seeded tissue. In certain embodiments, the flexible material 1304 is continuous and covers substantially the entire wire 1304. In yet other embodiments, the flexible material 1304 covers only a portion of the wire 902, such as selected portions of the circumference the wire 1302.

In certain embodiments, the flexible material 1304 includes a thickness that advantageously allows for the deformation for the wire 1302 from a first configuration to a second configuration. For example, the flexible material may comprise a thickness of between approximately 0.05 mm and approximately 0.762 mm.

As discussed above, in certain embodiments, the progress of the size change of the tissue shaping device 900 can be measured or monitored in real-time using conventional imaging techniques. Energy from conventional imaging devices can also be used to activate the shape memory material and change at least one dimension of the tissue shaping device 1300.

Furthermore, the tissue shaping device 1300 may comprise two or more sections or zones of shape memory material having different temperature response curves. For example, the wire 1302 may comprise at least two different shape memory materials.

FIG. 9B is an enlarged perspective view of a portion of a tissue shaping device 1350 including a first wire 1352 and a second wire 1354. Also illustrated are a first coating 1356, a second coating 1358 and a flexible material 1360, portions of which are shown removed to expose the first wire 1352 and the second wire 1354.

In certain embodiments, the first wire 1352 and second wire 1354 advantageously include shape memory materials that have different properties. For example, the first wire 1352 may respond to lower temperatures than the second wire 1354. Such embodiments advantageously allow the tissue shaping device 1350 to be adjusted to multiple configurations. For example, if each of the wires 1352, 1354 include two shape memory states or configurations, the tissue shaping device 1350 is capable of adjusting between four states or configurations.

In certain embodiments, the tissue shaping device 1350 is capable of contracting and expanding. For example, as discussed above, after the tissue shaping device 1350 has contracted, it may become necessary to expand the tissue shaping device 1350. For instance, the tissue shaping device 1350 may be implanted in a child with an enlarged heart. When the size of the heart begins to recover to its natural size, and the mitral valve reforms to its generally normal shape, the tissue shaping device 1350 can be adjusted. Then, as the child gets older and the heart begins to grow, the tissue shaping device 1350 can be further adjusted or removed from the heart as needed. In such certain embodiments, the first wire 1352 may be configured to contract the tissue shaping device 1350 and the second wire 1354 may be configured to expand the tissue shaping device 1350.

With continued reference to FIG. 13B, the outside surface of the first wire 1352 is substantially enclosed by the first coating 1356, and the outside surface of the second wire 1354 is substantially enclosed by the second coating 1358. In certain embodiments, the first coating 1356 and the second coating 1358 each comprise silicone tubing.

In certain other embodiments, the first coating 1356 and the second coating 1358 each comprise an energy absorption material, such as the energy absorption materials discussed above. In certain embodiments, the first coating 1356 heats when exposed to a first form of energy, and the second coating 1358 heats when exposed to a second form of energy. For example, the first coating 1356 may heat when exposed to MRI energy, and the second coating 1358 may heat when exposed to HIFU energy. As another example, the first coating 1356 may heat when exposed to RF energy at a first frequency, and the second coating 1358 may heat when exposed to RF energy at a second frequency. Thus, the first wire 1352 and the second wire 1354 can be activated independently such that one transitions to its austenite phase while the other remains in its martensite phase.

As also shown, the first and second wires 1352, 1354 and respective coatings 1356, 1358 are covered by the flexible material 1360, which may be similar to the flexible coating 1304 depicted in FIG. 13A. In certain embodiments, the flexible material 1312 operatively couples the first wire 1352 and the second wire 1354 such that a shape change in one mechanically affects the shape of the other. As discussed above, the first and second wires 1352, 1354 may each comprise a different shape memory material, such as the shape memory materials discussed above, that are activated at different temperatures.

In certain embodiments, the tissue shaping device 1350 is heated to a first temperature that causes the first wire 1352 to transition to its austenite phase and contract to its memorized shape. At the first temperature, the second wire 1354 is in its martensite phase and is substantially flexible as compared to the contracted first wire 1352. Thus, when the first wire 1352 transitions to its austenite phase, it exerts a sufficient force on the second wire 1354 through the flexible material 1360 to deform the second wire 1354 and cause the tissue shaping device 1350 to change shape.

The tissue shaping device 1350 can be expanded by heating the tissue shaping device to a second temperature that causes the second wire 1354 to transition to its austenite phase and expand to its memorized shape. In certain embodiments, the second temperature is higher than the first temperature. Thus, at the second temperature, both the first and second wires 1352, 1354 are in their respective austenite phases.

In certain embodiments, the diameter of the second wire 1354 is sufficiently larger than the diameter of the first wire 1352 such that the second wire 1354 exerts a greater force to maintain its memorized shape in the austenite phase than the first wire 1352. Thus, the first wire 1352 is mechanically deformed by the force of the second wire 1354 and the tissue shaping device 1350.

In certain embodiments, the first wire 1352 is configured to contract when transitioning to its austenite phase. In certain such embodiments, the first wire 1352 has an austenite start temperature Aₛ in a range between approximately 33° C. and approximately 43° C., an austenite finish temperature A_{f} in a range between approximately 45° C. and approximately 55° C., a martensite start temperature Mₛ less than approximately 30° C., and a martensite finish temperature M_{f} greater than approximately 20° C. In other embodiments, the austenite finish temperature A_{f} of the first wire 1352 is in a range between approximately 48.75° C. and approximately 51.25° C.

In certain embodiments, the second wire 1354 is configured to expand when transitioning to its austenite phase. In certain such embodiments, the second wire 1354 has an austenite start temperature Aₛ in a range between approximately 60° C. and approximately 70° C., an austenite finish temperature A_{f} in a range between approximately 65° C. and approximately 75° C., a martensite start temperature Mₛ less than approximately 30° C., and a martensite finish temperature M_{f} greater than approximately 20° C. In other embodiments, the austenite finish temperature A_{f} of the first wire 1352 is in a range between approximately 68.75° C. and approximately 71.25° C.

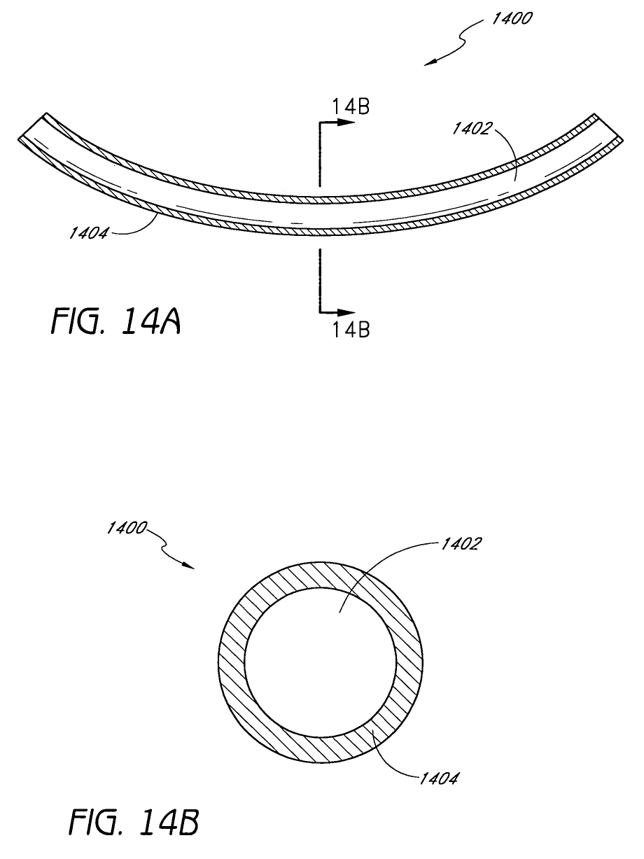

FIG. 14A illustrates a tissue shaping device 1400 including a shape memory wire 1402 substantially coated with an energy absorption layer 1404 according to certain embodiments. As discussed above, the energy absorption layer 1404 advantageously enhances energy absorption by other materials, such as the wire 1402. For example, the energy absorption layer 1404 may comprise at least one material and/or structure used to absorb energy from, for example, HIFU, MRI, inductive heating, combinations of the same or the like. In certain embodiments, the energy absorption layer 1404 increases heating efficiency and localizes heating in particular areas of the shape memory wire 1402 such that damage to surrounding tissue is reduced or minimized.

FIG. 14B illustrates a cross-sectional view of the tissue shaping device 1400. In particular, the energy absorption layer 1404 is shown as surrounding the outside surface of the shape memory wire 1402. In other embodiments, the energy absorption layer 1404 may comprise multiples layers for improving absorption of energy. For example, different layers may be capable of responding to different types of energy. In certain other embodiments, the energy absorption layer 1404 covers only a portion of the outside surface of the wire 1402, or the energy absorption material may be located within the wire 1402.

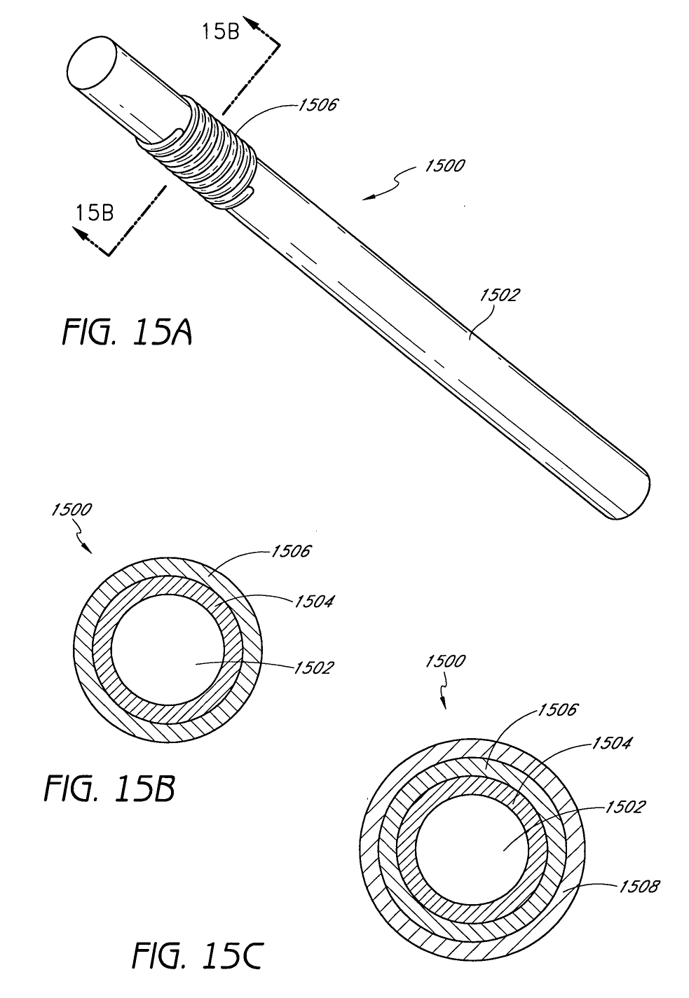

FIG. 15A illustrates a tissue shaping device 1500 including an electrically conductive coil 1506 according to certain embodiments. In one embodiment, the tissue shaping device 1500 is similar to the tissue shaping device 1400 of FIGS. 14A and 14B and comprises a shape memory wire 1502 responsive to changes in temperature as discussed above.

In certain embodiments, the electrically conductive coil 1506 comprises copper, gold, titanium, platinum, platinum iridium, stainless steel, ELGILOY®, alloys or combinations of the same or the like.

FIG. 15B illustrates a cross-sectional view of the tissue shaping device 1500. In particular, illustrated coil 1506 surrounds an energy absorption layer 1504 (not shown in FIG. 15A) that covers a shape memory wire 1502, which may be similar to the energy absorption layer 1404 and wire 1402 discussed above.

With reference to FIG. 15A, the illustrated coil 1506 is wrapped around a portion of the wire 1502 where it is desired to focus energy and heat the tissue shaping device 1500. In certain embodiments, the coil 1506 is wrapped around approximately 5% to approximately 15% of the wire 1502. In other embodiments, the coil 1506 is wrapped around approximately 15% to approximately 70% of the wire 1502. In other embodiments, the coil 1506 is wrapped around substantially the entire wire 1502. In certain embodiments, the tissue shaping device 1500 may include the energy absorption layer 1504 only between the coil 1506 and the wire 1502 and/or on portions of the wire 1502 not wrapped by the coil 1506. In yet other embodiments, the tissue shaping device 1500 may function without the energy absorption layer 1504.

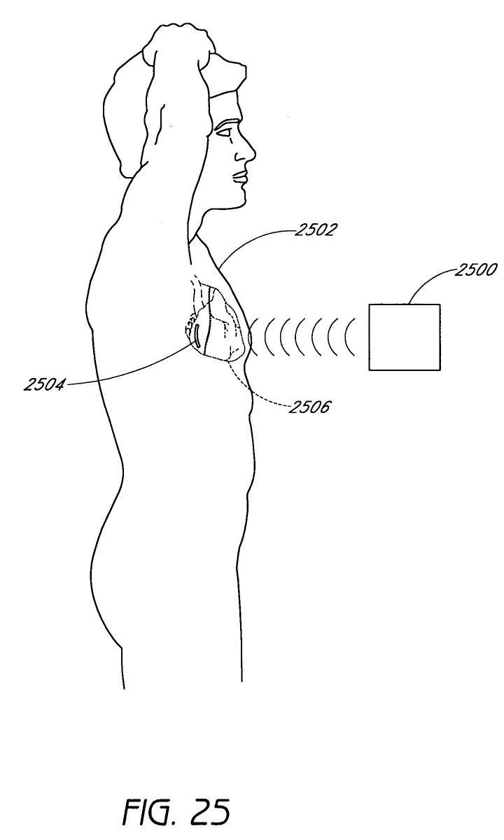

In certain embodiments, an electric current is non-invasively induced in the coil 1506 using electromagnetic energy. For example, in certain embodiments, a handheld or portable device comprising an electrically conductive coil, which is described in more detail with respect to FIG. 25, generates an electromagnetic field that non-invasively penetrates the patient's body and induces a current in the coil 1506. This electric current, in turn, causes the coil 1506 to heat. The coil 1506, the wire 1502 and the coating 1504 (if any) are advantageously thermally conductive such that heat or thermal energy transfers from the coil 1506 to the wire 1502. Thus, thermal energy can be directed to the wire 1502, or portions thereof, while reducing thermal damage to surrounding tissue.

FIG. 15C further illustrates the tissue shaping device 1500 according to certain embodiments as including an outer layer 1508. The outer layer 1508 comprises at least one material for facilitating medical procedures using the tissue shaping device 1500. In certain embodiments, the outer layer 1508 substantially envelops the entire tissue shaping device 1500. In other embodiments, the outer layer 1508 covers only a portion of the tissue shaping device 1500.

In certain embodiments, the outer layer 1508 comprises a lubricious material that facilitates placement of the tissue shaping device 1500 within the myocardial tissue 304. In certain such embodiments, the lubricious material is hydrogel or TEFLON®. In other embodiments, the lubricious material may comprise surface treated silicone or polyurethane materials, combinations of the same or the like.

In addition or in others embodiment, the outer layer 1508 comprises an anti-inflammatory coating to decrease inflammation response by the body of the patient. In certain such embodiments, the anti-inflammatory coating is Dexamethasone sodium phosphate or Dexamethasone sodium acetate. In other embodiments, the anti-inflammatory coating may comprise heparin or the like.

In certain embodiments, the outer layer 1508 advantageously encapsulates at least a portion of the coil 1506 and/or wire 1502 such that they do not contact tissue or fluid of the patient. For example, the outer layer 1508 may advantageously comprise a biocompatible, flexible material, such as, for example, a polyurethane tube. In other embodiments, the outer layer 1508 may comprise polytetrafluoroethylene (“TEFLON®”) or expanded polytetrafluoroethylene (ePTFE). In yet other embodiments, the outer layer 1508 may comprise DACRON®, woven velour, heparin-coated fabric, bovine or equine pericardium, homograft, patient graft, cell-seeded tissue, combinations of the same or the like.

In other embodiments, the outer layer 1508 comprises a biodegradable jacket or sleeve that facilities removal of the tissue shaping device 1500 from the myocardial tissue 304. For example, once physical remodeling of the mitral valve 102 has taken place (as determined, for example, by viewing Doppler enhanced echocardiograms), the tissue shaping device 1500 may be removed while the outer layer 1508 remains within the myocardial tissue 304. In certain embodiments, the outer layer 1508 advantageously comprises a polylactic acid (PLA). In other embodiments, the outer layer 1508 jacket comprises poly vinyl alcohol (PVA) or the like. In yet other embodiments, the outer layer 1508 comprises multiple layers, such as, for example, a biocompatible inner layer and a biodegradable outer layer.

In certain embodiments, the tissue shaping devices disclosed herein may also comprise thermal conductors usable to mark desired locations of the tissue shaping device. For example, the thermal conductors may be disposed at locations on the tissue shaping device corresponding to at least one commissure of the leaflets 204, 206. As another example, the thermal conductors may be used to align a percutaneous energy source, such as a heated balloon inserted through a catheter, with the tissue shaping device. In certain embodiments, the thermal conductors comprise materials such as gold, copper or other like imaging materials.

As described previously, in certain embodiments, the tissue shaping devices discussed herein may be advantageously and dynamically adjusted in a non-invasive manner through an energy source located external to the patient's heart. FIG. 25 illustrates a schematic view of an external source 2500 usable outside a patient's body 2502 to adjust a tissue shaping device 2504 positioned within a heart 2506. The external source 2500 includes any transducer, transmitter or the like capable of transmitting energy to the tissue shaping device 2504 and usable to effectuate a change in the shape and/or size of the tissue shaping device 2504.

As described previously, the external source 2500 may include an electrically conductive coil for generating an electromagnetic field that non-invasively penetrates the patient's body 2502 and induces a current in the tissue shaping device 2504. In other embodiments, the external source 2500 includes an external HIFU transducer that focuses ultrasound energy onto the tissue shaping device 2504. In yet other embodiments, the external source 2500 is configured to transmit, for example, radio frequency (RF) energy, x-ray energy, microwave energy, acoustic energy, light energy, electric field energy, magnetic field energy, combinations of the foregoing, or the like to the tissue shaping device 2504.

For example, in certain embodiments, the tissue shaping device 2504 includes at least one electromagnet. In such an embodiment, the external source 2500 may comprise an electromagnetic transmitter, such as a resistive coil, usable to activate the electromagnet(s) to cause a change in shape of the tissue shaping device 2504. Such a shape change may be used to adjust at least one dimension of the mitral valve annulus. For instance, the tissue shaping device 2504 may include an electromagnet on a first end and a magnetic material on a second end. As the external source 2500 emits a field to activate the electromagnet, the electromagnet attracts or repels the magnetic material, thus causing a change in the shape of the tissue shaping device 2504.

Implantation of Tissue Shaping Devices

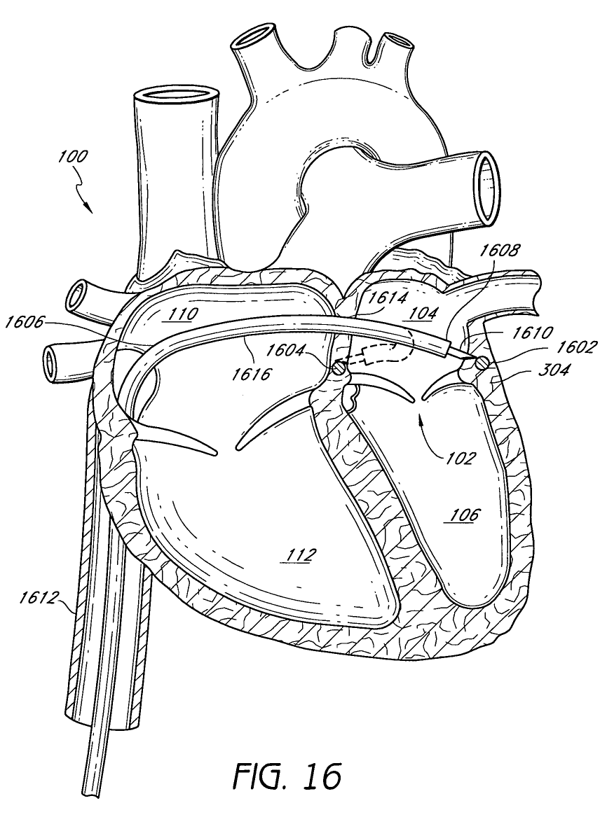

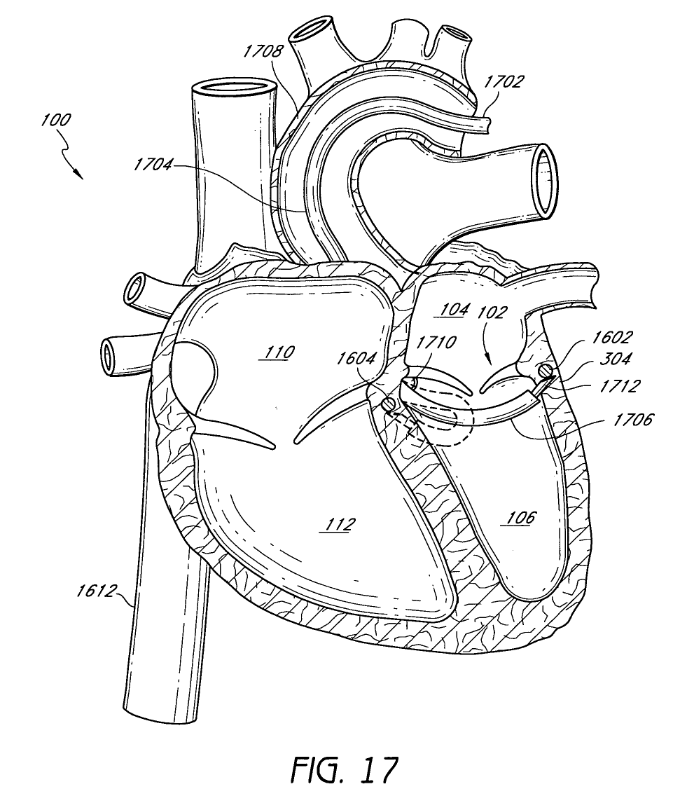

The tissue shaping devices disclosed herein may be implanted into a patient surgically, endoscopically, and/or percutaneously via a catheter delivery system. For example, FIGS. 16 and 17 depict exemplary methods usable to implant tissue shaping devices 1602, 1604 within myocardial tissue 304 according to certain embodiments. The tissue shaping devices 1602, 1604 are shown as round or disc-shaped devices. However, an artisan will recognize that the tissue shaping devices 1602, 1604 may have other configurations and may include, for example, the shapes, configurations, and/or magnetic and/or shape memory materials discussed above in relation to tissue shaping devices 302, 502, 900, 1100, 1200, 1300, 1350, 1400, or 1500.

FIG. 16 is a cross-sectional view of the human heart 100 shown in FIG. 1 and a distal portion of a transseptal delivery system 1606 using a transseptal approach to deliver the tissue shaping devices 1602, 1604. A distal end 1608 of the delivery system 1606, which in certain embodiments is deflectable from a proximal control mechanism (not shown), is engaged with the myocardial tissue 304 with a penetration member or moveable needle 1610 inserted into the myocardial tissue 304 as shown. The deflectability in combination with rotation of the delivery system allows the distal end 1608 of the delivery system 1606 to be positioned at a variety of desired locations within the left atrium 104, as indicated by the dashed lines shown in FIG. 16.

To access the left atrium 104, the delivery system 1606 is inserted through the inferior vena cava 1612 and passed through the fossa ovalis 1614 from the right atrium 110. The moveable needle 1610 percutaneously makes a path into the myocardial wall 304. The tissue shaping devices 1602, 1604 are then ejected or pushed from the needle 1610 by a stylet (not shown) within the needle 1610. Advantageously, this approach is useful for deployment of the tissue shaping devices 1602, 1604 on the atrial side of the mitral valve 102. In certain embodiments, the transseptal delivery system 1606 includes a catheter body 1616 having an outer transverse dimension or diameter in a range between approximately 7 French and approximately 9 French.

The general transseptal approach to the left atrium 104 from the right atrium 110 is well known and is used in Electrophysiology and Cardiology, particularly when a retrograde approach (discussed below) is contraindicated. In certain exemplary embodiments, a commercially available transseptal access system is used that includes a “Mullins™ Introducer Sheath” having a stainless steel Brockenbrough needle manufactured by the Medtronic® company. Similar devices are also made by St. Jude Medical®, Inc. The Brokenbrough needle is used to make the transseptal puncture while the Mullins introducer sheath/dilator set serves as a conduit for the needle and the catheters that go through it. The Brokenbrough curved needle is made up of an outer cannula and an inner stylet. The outer cannula is made of a thin walled tubing of a material such as stainless steel. The inner stylet is solid, much stiffer and closely fitting within the inner lumen of the cannula. In certain embodiments, the sharp tip of the stylet protrudes about 2 mm to about 3 mm from the distal end of the cannula.

FIG. 17 is a cross-sectional view of the human heart 100 shown in FIG. 1 and a distal portion of a retrograde delivery system 1702 using a retrograde approach to deliver the tissue shaping devices 1602, 1604 to the myocardial tissue 304 on the ventricular side of the mitral valve 102. In certain embodiments, the delivery system 1702 includes a catheter body 1704 having a distal end 1706 which is deflectable from a proximal controller (not shown). The deflectability allows the distal end 1706 of the retrograde system 1702 to be maneuvered about the left ventricle 106, or any other portion of the heart by rotation in combination with deflection, as can the transseptal delivery system 1606 discussed above. An alternate positioning of the distal end 1706 of the delivery system 1702 is shown by dashed lines in FIG. 17.

To access the left ventricle 106, the delivery system 1702 is advanced through the aorta 1708 and through the aortic valve 1710. The distal end 1706 of the system 1702 is engaged with the myocardial tissue 304 with a penetration member or moveable needle 1712 inserted into the tissue 304. Once the needle 1712 has been inserted into the tissue 304 of a desired location within the heart 100, the tissue shaping devices 1602, 1604 are deployed or pushed from the needle 1712 by a stylet (not shown). In certain embodiments, the catheter body 1704 has an outer transverse dimension or diameter in a range between approximately 7 French and approximately 9 French.

Leaflet Braces

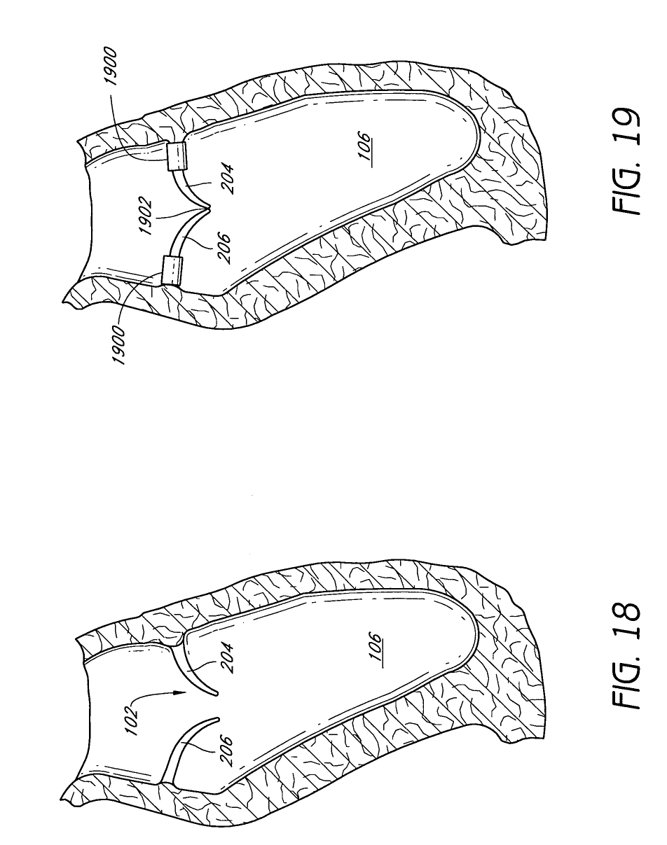

In other embodiments, valvular insufficiency is treated by directly reinforcing deformed valve leaflets. For example, FIG. 18 is a cross-sectional view of the left ventricle 106 of the heart shown in FIG. 1 wherein the leaflets 204, 206 of the mitral valve 102 are deformed such that proper sealing and valve function is impeded. FIG. 19 illustrates leaflet braces 1900 deployed over and crimped to the leaflets 204, 206 of the mitral valve 102 shown in FIG. 18. The leaflet braces 1900 mechanically support the valve leaflets 204, 206 and force the inward ends 1902 of the leaflets 204, 206 together for improved valve sealing and function.

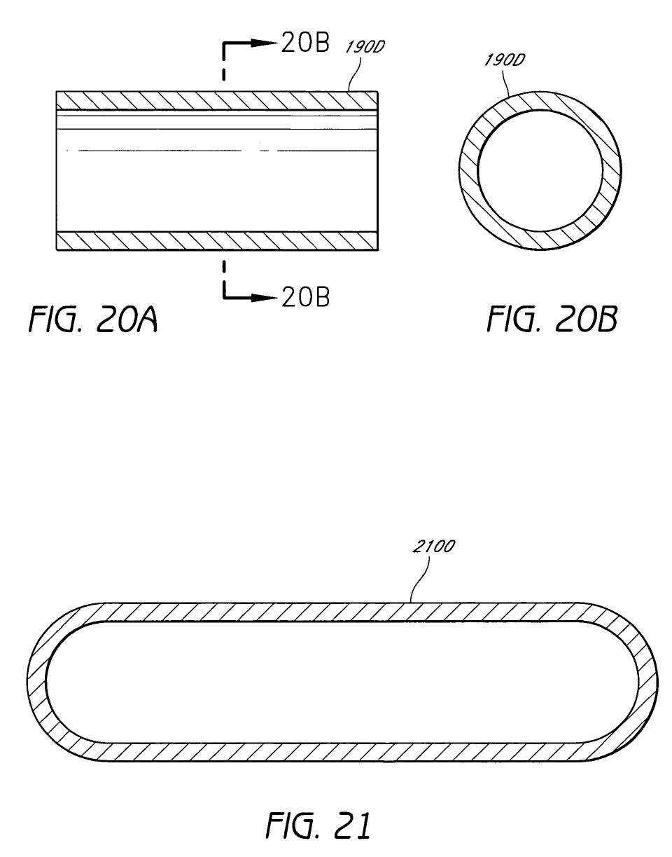

FIGS. 20A and 20B schematically illustrate the leaflet brace 1900 shown in FIG. 19 according to certain embodiments. The valve leaflet 1900 has a generally tubular configuration made from a high strength biocompatible material that is crimpable, such as stainless steel, NiTi, platinum iridium, gold, carbon, suitable polymers such as polyurethane, or the like. In certain embodiments, the brace 1900 has an axial length in a range between approximately 0.15 inches and approximately 0.250 inches and a transverse outer dimension or diameter in a range between approximately 0.13 inches and approximately 0.14 inches. In addition or in other embodiments, the leaflet brace 1900 has an inner transverse dimension or diameter in a range between approximately 0.08 inches and approximately 0.12 inches. In other embodiments, the brace 1900 has an inside transverse dimension or diameter in a range between approximately 0.020 inches and approximately 0.10 inches.

FIG. 21 schematically illustrates a transverse cross-section of a leaflet brace 2100 according to other embodiments. Advantageously, the leaflet brace 2100 has an elliptical or oval cross section that permits deployment at or near the base of the valve leaflets 204, 206. In certain embodiments, the leaflet brace 2100 has a transverse cross section width in a range between approximately 5 and approximately 10 times the height of the transverse cross section. In certain embodiments, the materials and dimensions of the brace 2100 are the same as or similar to the materials and dimensions of the brace 1900 discussed above.

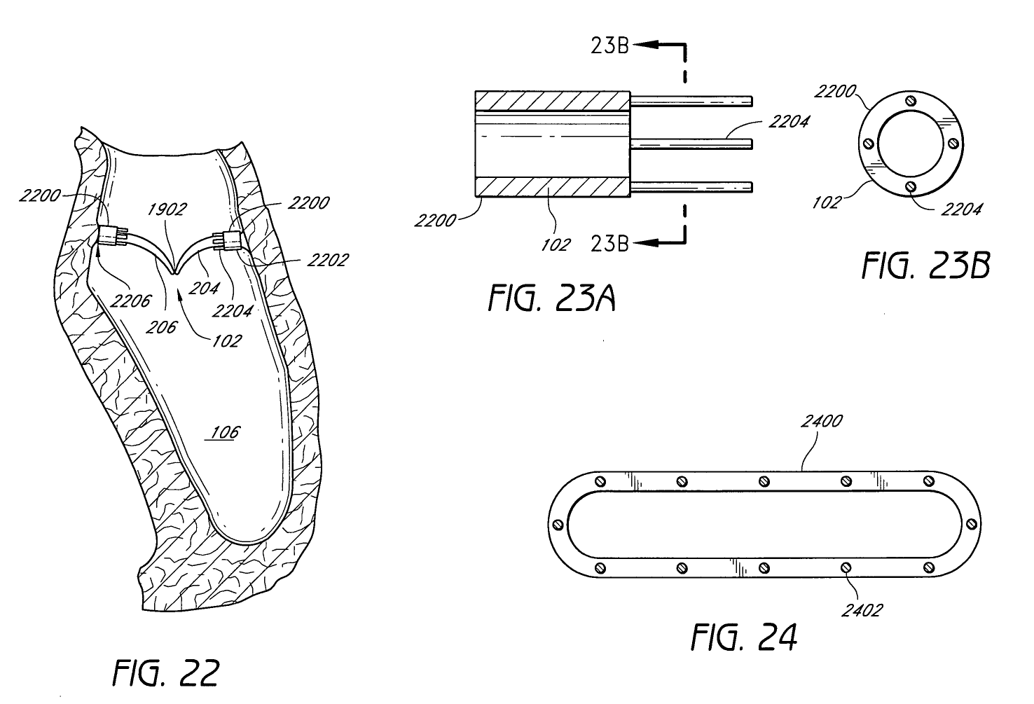

FIG. 22 is a cross-sectional view of the left ventricle 106 shown in FIG. 18 illustrating leaflet braces 2200 deployed over and crimped to the leaflets 204, 206 of the mitral valve 102 according to certain embodiments. The leaflet braces 2200 include a base portion 2202 and one or more resilient extensions 2204 extending axially from the base portion 2202. The base portion 2202 provides rigid mechanical strength and support to the base portion of the leaflets 204, 206 proximate the annular ring 2206 of the mitral valve 102.

The resilient extensions 2204 provide flexible support to the leaflets 204, 206 at the middle portions thereof. The rigid and flexible support provided by the braces 2200 urge the inward ends 1902 of the leaflets 204, 206 together for an improved seal and function. Thus, the braces 2200 advantageously prevent or reduce the regurgitation that would be present in the defective valve configuration as shown in the left ventricle 106 of FIG. 18. In certain embodiments, the braces 2200 are deployed by sliding the braces 2200 over the desired leaflet 204, 206 and crimping in place.

FIGS. 23A and 23B schematically illustrate the leaflet brace 2200 shown in FIG. 22 according to certain embodiments. As schematically illustrated, the base portion 2202 according to certain embodiments has a round transverse cross section and four resilient extensions 2204 extending in an axial direction from the base portion 2202. In such embodiments, the base portion 2202 has an axial length in a range between approximately 0.15 inches and approximately 0.25 inches, an inner transverse dimension or diameter in a range between approximately 0.005 inches and approximately 0.01 inches, and an outer transverse dimension in a range between approximately 0.13 inches and approximately 0.14 inches. In certain such embodiments, the flexible extensions 2204 have a length in a range between approximately 0.05 inches and approximately 0.1 inches, and an outer transverse dimension or diameter in a range between approximately 0.005 inches and approximately 0.010 inches.

In certain embodiments, the materials of the base portion 2202 and the flexible extensions 2204 are the same as or similar to the materials of the brace 1900 discussed above. In certain embodiments the flexible extensions 2204 advantageously comprise a flexible polymer material or fiber, such as carbon fiber.

FIG. 24 schematically illustrates a transverse cross-section of a leaflet brace 2400 according to other embodiments. Advantageously, the leaflet brace 2400 has a substantially oval or elliptical transverse cross section that permits deployment at or near the base of the valve leaflets 204, 206. The brace includes a plurality of flexible extensions 2402. In certain embodiments, the brace 2400 has a transverse cross section width in a range between approximately 5 and approximately 10 times the height of the transverse cross section. In certain embodiments, the materials and dimensions of the brace 2400 are the same as or similar to the materials and dimensions of the brace 1900 and/or the brace 2200 discussed above.

Resilient Tissue Shaping Devices

In certain embodiments, one or more tissue shaping devices comprising a resilient material are implanted in or near a heart valve annulus to reshape the heart tissue in the region adjacent the base of valve leaflets and improve leaflet coaptation. The heart tissue where the resilient tissue shaping devices may be deployed include, for example, myocardium, the interventricular septum of the heart, the left and/or right fibrous trigone, or a wall of the left or right atrium.

Advantageously, the resilient material can be mechanically strained so as to facilitate implantation into the heart tissue. After implantation, the strain is removed from the resilient material and the tissue shaping device recovers its pre-strained shape so as to reshape the surrounding heart tissue. In certain such embodiments, recovery of the pre-strained shape advantageously does not require the use of an external energy source to heat or otherwise activate the resilient material.

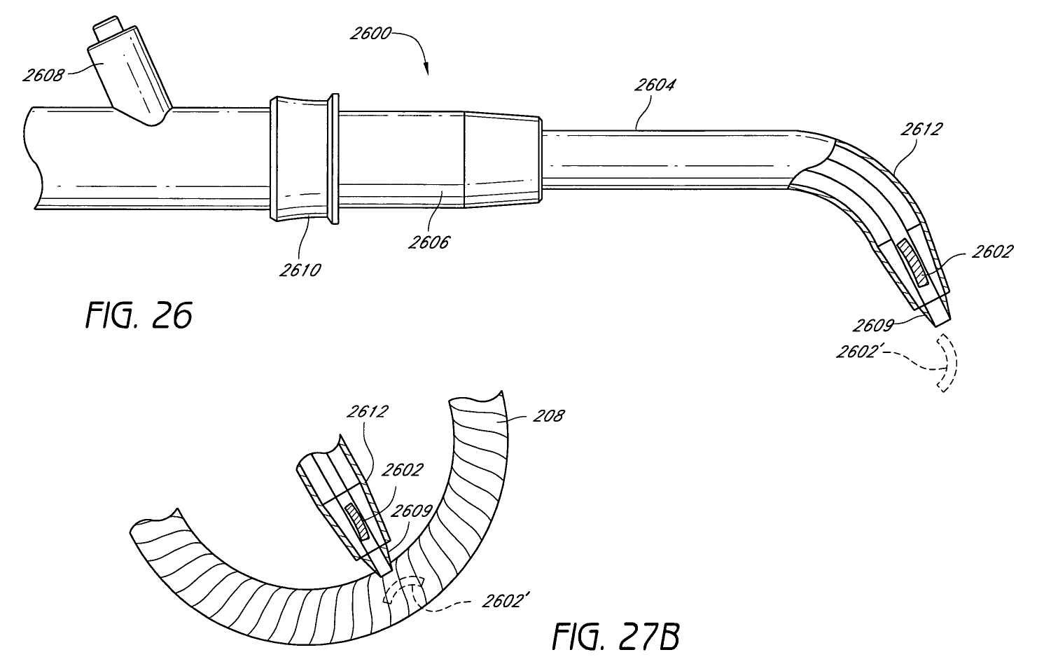

For example, FIG. 26 is a partial cross-sectional view of a catheter 2600 configured to deliver a resilient tissue shaping device 2602 according to certain embodiments. The catheter 2600 includes an elongate catheter body 2604 having a proximal end coupled to a handle 2606. The handle 2606 includes a side arm 2608 through which a hollow needle 2609 may be inserted and pushed through the catheter body 2604 so as to deliver the resilient tissue shaping device 2602 to heart tissue. In certain embodiments, the handle 2606 also includes a deflection controller 2610 configured to deflect a distal end 2612 of the elongate catheter body 2604.

In certain embodiments, the resilient tissue shaping device 2602 has an arcuate shape when it is not mechanically stressed, as illustrated by dashed lines as tissue shaping device 2602′. When inserted into the catheter 2600 through the side arm 2608, the resilient tissue shaping device 2602 is deformed into a less arcuate or substantially straight shape so as to fit within the catheter body 2604. The resilient tissue shaping device 2602 is then pushed through the elongate catheter body 2604 until it exits the distal end 2612 thereof. As the resilient tissue shaping device 2602 exits the distal end 2612, the stress caused by the catheter 2600 is removed and the resilient tissue shaping device 2602 recovers its arcuate shape, as shown by the dashed lines as tissue shaping device 2602′.

In certain embodiments, the resilient tissue shaping device 2602 comprises a metal or metal alloy capable of recovering its shape after deformation. In certain such embodiment, the resilient tissue shaping device 2602 comprises stainless steel configured to recover its shape after experiencing a strain in a range between as much as approximately 0.3% and approximately 0.8%.

In certain other embodiments, the resilient tissue shaping device 2602 comprises a shape memory material, such as the shape memory materials discussed above. Certain such shape memory materials have the ability to recover their shapes upon unloading after a substantial deformation. This property may be referred to as superelasticity or pseudoelasticity. In certain shape memory materials, superelasticity is based on stress-induced martensite formation when the shape memory material is above the austenite finish temperature (A_{f}). The application of an outer stress causes martensite to form at temperatures higher than the martensite start temperature (Mₛ). When the stress is released, the martensite transforms back into austenite and the shape memory material recovers its pre-stressed shape. In certain embodiments, the austenite finish temperature (A_{f}) and the martensite start temperature (Mₛ) are in the respective temperature ranges discussed above. In an exemplary embodiment, the austenite finish temperature (A_{f}) of the shape memory material is selected at or within a few degrees below a patient's body temperature. In certain embodiments, the resilient tissue shaping device 2602 comprises a NiTi alloy configured to recover its shape after experiencing a strain in a range between as much as approximately 8.0% and approximately 10.0%.

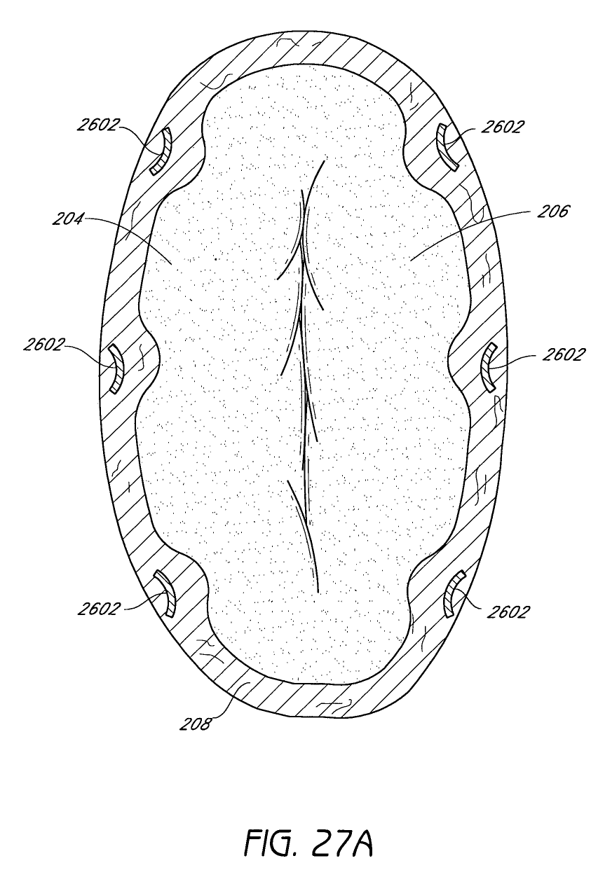

FIG. 27A illustrates a top schematic view of a plurality of resilient tissue shaping devices 2602 implanted in the mitral valve annulus 208 according to an exemplary embodiment. FIG. 27B schematically illustrates one of the resilient tissue shaping devices 2602 being implanted in the mitral valve annulus 208 through the distal end 2612 of the catheter 2600 and hollow needle 2609 shown in FIG. 26. As discussed above, the resilient tissue shaping device 2602 recovers its arcuate shape as it exits the needle 2609 and enters the mitral valve annulus 208. As the resilient tissue shaping devices 2602 recover their arcuate shapes upon implantation, they push the leaflets 204, 206 together to improve leaflet coaptation and reduce regurgitation.

While certain embodiments of the inventions have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. An implant for reinforcing a patient's heart valve, said implant comprising:
a body member having a proximal end, a distal end and a length extending therebetween, said body member configured to be implanted within a patient's heart at or near a base of a heart valve leaflet;
wherein said body member comprises a shape memory material and is transformable from a first configuration to a second configuration;
wherein, when said body member is in said second configuration, said body member is configured to reshape a tissue of the heart so as to exert a force on the leaflet base;
and wherein said implant is elongate with its longest length less than or equal to about fifteen millimeters.

2. The implant of claim 1, wherein said body member is substantially straight when in said first configuration.

3. The implant of claim 2, wherein said implant is implanted within said patient's heart when said body member is in said first configuration.

4. The implant of claim 2, wherein said body member comprises a substantially arcuate shape when in said second configuration.

5. The implant of claim 1, wherein said implant is configured to be implanted wholly within said tissue of the heart.

6. The implant of claim 1, wherein said implant is configured to be positioned adjacent a surface of said tissue of the heart.

7. The implant of claim 6, wherein said body member further comprises one or more anchor members configured to securely attach said body member to said surface of said tissue of the heart.

8. The implant of claim 1, wherein the longest length of said implant is less than or equal to about ten millimeters.

9. The implant of claim 1, wherein the longest length of said implant is less than or equal to about six millimeters.

10. The implant of claim 1, wherein said shape memory material is configured to be superelastic in at least one of said first configuration and said second configuration.

11. The implant of claim 1, wherein said heart tissue comprises myocardium.

12. The implant of claim 1, wherein said heart tissue comprises the interventricular septum of the heart.

13. The implant of claim 1, wherein said heart tissue comprises a fibrous trigone.

14. The implant of claim 1, wherein said heart tissue comprises a wall of an atrium.

15. The implant of claim 1, wherein said implant is configured to be deliverable by a retrograde delivery system utilizing a retrograde approach into the left ventricle of the patient's heart when said body member is in said first configuration.

16. The implant of claim 1, wherein said implant is configured to be deliverable by a transseptal delivery system utilizing a transseptal approach into the left atrium of the patient's heart when said body member is in said first configuration.

17. The implant of claim 1, wherein said shape memory material comprises a shape memory alloy.

18. The implant of claim 1, wherein said shape memory material comprises a shape memory polymer.

19. The implant of claim 1, wherein said shape memory material is ferromagnetic.

20. The implant of claim 19, wherein said shape memory material comprises at least one of Fe—C, Fe—Pd, Fe—Mn—Si, Co—Mn, Fe—Co—Ni—Ti, Ni—Mn—Ga, Ni₂MnGa, and Co—Ni—Al.

21. The implant of claim 20, wherein said body member is configured to transform from said first configuration to said second configuration without substantially changing the temperature of said ferromagnetic shape memory material.

22. The implant of claim 1, wherein said body member is configured to transform from said first configuration to said second configuration when said shape memory material is activated by an energy source.

24. The implant of claim 22, further comprising an energy absorption enhancement material configured to absorb energy in response to said energy source, said energy absorption enhancement material in thermal communication with said shape memory material.

25. The implant of claim 24, wherein said energy absorption enhancement material comprises a nanoparticle.

26. The implant of claim 25, wherein said nanoparticle comprises at least one of a nanoshell and a nanosphere.

27. The implant of claim 24, wherein said energy absorption enhancement material is radiopaque.

28. The implant of claim 24, wherein said energy absorption enhancement material is further configured to heat in response to said energy source.

29. The implant of claim 22, further comprising an electrically conductive material configured to conduct a current in response to the energy source and to transfer thermal energy to the shape memory material.

30. An implant for reinforcing a patient's heart valve, said implant comprising:
a body member having a proximal end, a distal end and a length extending therebetween;
wherein said body member comprises a shape memory material and is transformable from a first configuration to a second configuration;
wherein, when said body member is in said second configuration, said body member is configured to reshape a tissue of the heart so as to exert a force on the leaflet base;
and wherein said implant is elongate and is configured to be wholly implanted within said heart tissue.

31. The implant of claim 30, wherein said implant is substantially straight when said body member is in said first configuration.

32. The implant of claim 31, wherein said implant is implanted within said patient's heart when said body member is in said first configuration.

33. The implant of claim 31, wherein said implant comprises a substantially arcuate shape when said body member is in said second configuration.

34. The implant of claim 30, wherein the longest length of said implant is less than or equal to about fifteen millimeters.

35. The implant of claim 30, wherein the longest length of said implant is less than or equal to about ten millimeters.

36. The implant of claim 30, wherein the longest length of said implant is less than or equal to about six millimeters.

37. The implant of claim 30, wherein said shape memory material is configured to be superelastic in at least one of said first configuration and said second configuration.

38. A method of treating heart valve disease, comprising:
providing an implant comprising a body member having a proximal end, a distal end and a length extending therebetween, wherein said body member comprises a shape memory material;
wholly implanting said implant within a tissue of a patient's heart at or near a base of a valve leaflet; and
applying energy to said shape memory material so as to transform said implant from a first configuration having a first shape to a second configuration having a second shape.

39. The method of claim 38, wherein said implant in said second configuration reshapes tissue adjacent said implant and produces a change in a dimension of the annulus of the valve.

40. The method of claim 39, wherein said change in dimension urges the base of the leaflet toward the center of the heart valve.

41. The method of claim 38, wherein applying said energy comprises applying said energy with an energy source located outside the patient's heart and unattached to said implant.

42. The method of claim 38, wherein positioning said implant comprises delivering said implant using a retrograde approach through the patient's aorta into the left ventricle of the patient's heart.

43. The method of claim 38, wherein positioning said implant comprises delivering said implant using a transseptal approach into the left atrium of the patient's heart.

44. A device for reshaping or reforming body tissue, the device comprising:
resilient means for changing a dimension of a heart valve annulus, said resilient means configured to be implanted at or near the base of a leaflet of a patient's heart valve, said resilient means configured to transform from a first shape to a second shape in response to a force applied thereto during implantation, wherein said resilient means transforms back to said first shape when said force is removed therefrom after said implantation.

45. The device of claim 44, wherein said resilient means is configured to be wholly implanted within a tissue of the heart.

46. A method for changing a dimension of a heart valve annulus, said method comprising:
implanting a first device in a patient's heart, wherein said first device is magnetic;
implanting a second device in said patient's heart;
wherein said second device is responsive to a magnetic field emanating from said first device so as to produce a change in a dimension of a heart valve annulus.

47. The method of claim 46, wherein said change in said dimension comprises a decrease.

48. The method of claim 46, wherein said second device is magnetic.

49. The method of claim 48, wherein said magnetic field is a first magnetic and wherein said first device is responsive to a second magnetic field emanating from said second device so as to further produce said change in said dimension of the heart valve annulus.

50. The method of claim 46, wherein said first device is implanted adjacent a first leaflet of the heart valve and said second device is implanted adjacent a second leaflet of the heart valve such that said second device's response to said magnetic field urges the base of the second leaflet toward the base of the first leaflet.

51. The method of claim 46, wherein said first device is implanted on the atrial side of the heart valve annulus adjacent a first leaflet thereof and said second device is implanted on the ventricular side of the heart valve annulus adjacent the first leaflet, and wherein said second device's response to said magnetic field urges the base of the first leaflet toward a base of a second leaflet of the heart valve.

52. The method of claim 46, wherein at least one of said first device and said second device is implanted within myocardial tissue.

53. The method of claim 46, wherein at least one of said first device and said second device is implanted on a surface of a tissue of the heart using one or more anchor members.

54. The method of claim 46, further comprising electrically activating at least one of said first device and said second device.

55. The method of claim 54, wherein said electrically activating comprises activating at least of said first device and said second device with an electromagnetic transmitter located outside the heart.

56. A tissue shaping system comprising:
a first device configured to emanate a magnetic field, said first device configured to be implanted at or near a heart valve annulus; and
a second device configured to interact with said first device by responding to said magnetic field, said second device configured to be implanted at or near the heart valve annulus;
wherein said first device is configured to interact with said second device so as to change a dimension of the heart valve annulus.

57. The tissue shaping system of claim 56, wherein said second device is magnetic.

58. The tissue shaping system of claim 57, wherein said interaction between said first device and said second device is an attraction.

59. The tissue shaping system of claim 56, wherein said first device and said second device are configured to exert at least one force sufficient to decrease said dimension of the heart valve annulus when said first device and said second device are implanted adjacent thereto.

60. The tissue shaping system of claim 56, wherein said first device comprises a rare earth element.

61. The tissue shaping system of claim 56, wherein said first device comprises at least one of the following: NdFeB (Neodymium Iron Boron), SmCo (Samarium Cobalt) and AlNiCo (Aluminum Nickel Cobalt).

62. The tissue shaping system of claim 56, further comprising at least one fixation member configured to anchor at least one of said first device and said second device to the heart valve annulus.

63. A system for reshaping or reforming a heart valve annulus, said system comprising:
means for emanating a magnetic field; and
means for interacting with said means for emanating by responding to said magnetic field;
wherein, when said means for emanating and said means for interacting are implanted at or near the heart valve annulus, at least one dimension of the heart valve annulus is changed while said means for interacting responds to said magnetic field.
